(19) United States
(12) Patent Application Publication
(10) Pub. No.: US 2025/0051301 A1
(43) Pub. Date: Feb. 13, 2025

(54) MUTANT IDH1 AND IDH2 INHIBITOR AND APPLICATION THEREOF

(71) Applicant: Betta Pharmaceuticals Co., Ltd, Hangzhou (CN)

(72) Inventors: Yabin Li, Beijing (CN); Xiaofeng Xu, Beijing (CN); Xizhen Song, Beijing (CN); Jingwei Ding, Beijing (CN); Yunlai Zhang, Hangzhou (CN); Jie Chen, Beijing (CN); Xiangyong Liu, Beijing (CN); Lieming Ding, Hangzhou (CN); Jiabing Wang, Beijing (CN)

(21) Appl. No.: 18/847,130

(22) PCT Filed: Mar. 14, 2023
(86) PCT No.: PCT/CN2023/081225

§ 371 Date: Sep. 13, 2024

(30) Foreign Application Priority Data
Mar. 15, 2022 (CN) 202210252930.0

(51) Int. Cl. C07D 401/04; A61K 31/53

(52) U.S. Cl. CPC C07D 401/04; A61K 31/53

(57) ABSTRACT

Disclosed are a compound represented by formula (I) as a mutant isocitrate dehydrogenase 1 and 2 (IDH1 and IDH2) inhibitor, a preparation method therefor, and a pharmaceutical composition thereof. Also disclosed is a use of the described compound or the pharmaceutical composition thereof in the treatment of mutant IDH1 and IDH2-mediated diseases.

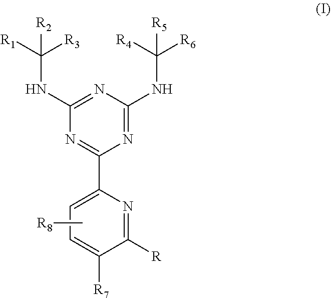

### TECHNICAL FIELD

The present invention relates to a series of compounds as mutant isocitrate dehydrogenase 1 and 2 (IDH1 and IDH2) inhibitors, preparation methods therefor, and pharmaceutical compositions thereof. The present invention also relates to the use of the above-mentioned compounds or pharmaceutical compositions thereof in the treatment of mutant IDH1 and IDH2-mediated diseases.

### BACKGROUND ART

Three metabolic enzymes, namely fumarate dehydrogenase, succinate dehydrogenase and isocitrate dehydrogenase (IDH), have been found in tumor cells in recent years. Genetic mutations in these enzymes alter cellular metabolism and may be associated with the occurrence and development of tumors.

In humans, there are three types of IDHs, namely IDH1, IDH2 and IDH3. IDH1 is localized in cytosol and peroxisomes, whereas IDH2 and IDH3 are both localized in mitochondria. Such proteases can oxidize isocitrate to oxalosuccinate, which is then converted to α-ketoglutarate (α-KG).

In 2008, IDH1 gene mutations were inadvertently discovered during gene sequencing in human cerebral glioblastoma, which opened the door to IDH research in the field of tumors. Subsequently, it has been found in several large-scale clinical case-control studies on glioma that IDH1 gene mutations are prevalent in over 75% of low-grade glioma and 90% of secondary glioblastoma; and IDH2 gene mutations are prevalent in approximately 20% of acute myeloid leukemia. Furthermore, IDH gene mutations have also been reported in cholangiocarcinoma (10% to 23%), melanoma (10%) and chondroid tumors (75%). It can be seen therefrom that IDH mutations can be found in a variety of tumors. Common mutation sites include arginine residues located in the catalytic center (IDH1/R132H, IDH1/R132C, IDH2/R140Q and IDH2/R172K). The mutated IDH can catalyze the conversion of α-ketoglutarate (α-KG) to 2-hydroxyglutaric acid (2-HG). Studies have shown that α-KG and 2-HG are similar in structure, and 2-HG competes with α-KG, thereby reducing the activity of α-KG-dependent enzymes and leading to hypermethylation of chromatin. Such hypermethylation is believed to interfere with normal cell differentiation and lead to excessive proliferation of immature cells, thus inducing cancer.

Agios Pharmaceuticals published their findings in Science in 2013, and showed that the mutant IDH1 enzyme inhibitor AGI-5198 (Science, 2013, 340, 626-630) and mutant IDH2 enzyme inhibitor AGI-6780 (Science, 2013, 340, 622-626) developed by the company can effectively inhibit the production of 2-HG mediated by mutant IDH1/IDH2 in cells and induce the differentiation of abnormally proliferating cancer cells. The treatment of glioma cells carrying IDH1 gene mutations with AGI-5198 and the treatment of leukemia cells carrying IDH2 gene mutations with AGI-6780 both resulted in increased expression of maturation markers in the cells.

Phase I clinical trials of AG-120, a mutant IDH1 inhibitor developed by Agios Pharmaceuticals, showed that in acute myeloid leukemia (AML) or myelodysplastic syndrome (MDS) patients carrying IDH1 gene mutations, a decrease in α-hydroxyglutaric acid (2-HG) levels was observed in 98% of the patients.

In 2013, Agios Pharmaceuticals reported the IDH2^{R140Q} inhibitor AGI-6780 and the IDH2^{R132H} inhibitor AGI-5198, as well as another IDH2^{R140Q} inhibitor AG-221, which was later marketed by the company. AGI-6780 and AGI-5198 can respectively inhibit the production of 2-HG in cells carrying common IDH1 and IDH2 mutants.

Agios Pharmaceuticals also applied for patent WO 2015003640A1 in 2014, disclosing an IDH1 and IDH2 inhibitor

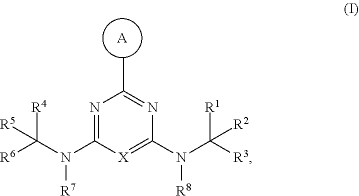

In 2017, Eli Lilly and Company reported an IDH1 and IDH2 inhibitor

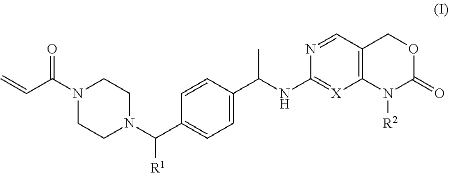

and applied for patent WO 2018111707A1. In 2019, Hutchison Whampoa Limited

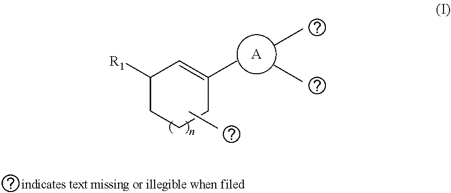

applied for patent WO 2019047909A1 for an IDH1 and IDH2 inhibitor.

For cancers caused by IDH1 and IDH2 mutations, such as brain glioma, acute myeloid leukemia, cholangiocarcinoma and melanoma, AG120 (IDH1 single inhibitor) and AG221 (IDH2 single inhibitor) are currently on the market, providing medication options for clinical use. New studies have found that IDH1 and IDH2 mutations may coexist in the same tumor, which results in that an IDH1 or IDH2 single inhibitor has limited efficacy and develop acquired drug resistance. Although drugs for treating cancers by simultaneous inhibition of mutant IDH1 and IDH2 have been studied and reported, there remains a need to develop a novel dual inhibitor of mutant IDH1 and IDH2 with strong target inhibitory ability and excellent selectivity for use in the treatment of related mutant IDH1 and IDH2-mediated diseases, so as to overcome the problem of acquired resistance caused by long-term administration of a single inhibitor and provide a new medication option for clinical use.

### SUMMARY

The present invention relates to a compound represented by formula (I), or a pharmaceutically acceptable salt, solvate, chelate, non-covalent complex or prodrug thereof, wherein the compound represented by formula (I) has the following structure:

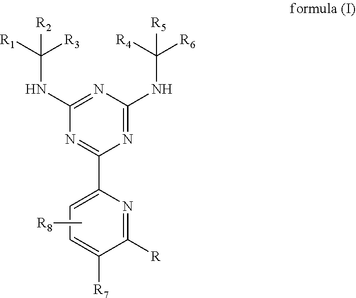

wherein
R is selected from halogen, —NO₂, —CN, —NH₂, C₁-C₄ alkyl and C₁-C₄ haloalkyl;
R₁, R₂, R₃, R₄, R₅ and R₆ are the same or different, and are each independently selected from hydrogen, halogen, —CN, C₁-C₄ alkyl, —O—C₁-C₄ alkyl, C₃-C₆ cycloalkyl and 3-6 membered heterocyclyl; the C₁-C₄ alkyl, C₃-C₆ cycloalkyl and 3-6 membered heterocyclyl are optionally substituted with one or more of hydrogen, halogen, oxo, —NO₂, —OH, —NH₂, —CN, C₁-C₄ alkyl, —O—C₁-C₄ alkyl, —NH(C₁-C₄ alkyl) or —N(C₁-C₄ alkyl)₂;
R₂ and R₃ optionally together with the carbon atom to which they are attached form C(═O); or
R₅ and R₆ optionally together with the carbon atom to which they are attached form C(═O); or
R₂ and R₃ optionally together with the carbon atom to which they are attached form C₃-C₁₀ cycloalkyl and 3-10 membered heterocyclyl, wherein the C₃-C₁₀ cycloalkyl and 3-10 membered heterocyclyl are optionally substituted with one or more of —OH, —NH₂, —CN, halogen, oxo, —NO₂, C₁-C₄ alkyl and C₁-C₄ alkoxy; or
R₅ and R₆ optionally together with the carbon atom to which they are attached form C₃-C₁₀ cycloalkyl and 3-10 membered heterocyclyl, wherein the C₃-C₁₀ cycloalkyl and 3-10 membered heterocyclyl are optionally substituted with one or more of —OH, —NH₂, —CN, halogen, oxo, —NO₂, C₁-C₄ alkyl and C₁-C₄ alkoxy;
R₇ is selected from halogen, —OR₉, C₁-C₄ alkyl, C₁-C₄ haloalkyl or —N(R₉)₂;
R₈ is selected from hydrogen, halogen, —NO₂, —CN, Cr C₄ alkyl, C₂-C₄ alkenyl, —OR₉ or —N(R₉)₂, wherein the C₁-C₄ alkyl and C₂-C₄ alkenyl are optionally substituted with one or more of hydrogen, halogen, oxo, —NO₂, —OH, —NH₂ and —CN; and
each R₉ is the same or different, and is independently selected from hydrogen and C₁-C₄ alkyl, wherein the C₁-C₄ alkyl is optionally substituted with one or more of hydrogen, halogen, oxo, —NO₂, —OH, —NH₂ and —CN.

In some embodiments, the compound represented by formula (I), or the pharmaceutically acceptable salt, solvate, chelate, non-covalent complex or prodrug thereof is selected from a compound having a structure represented by formula (11):

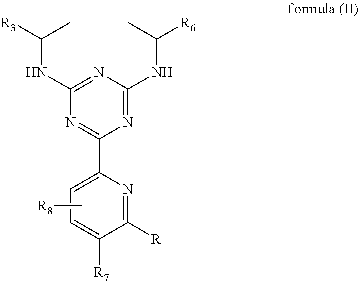

wherein
R is selected from halogen, —NO₂, —CN, —NH₂, C₁-C₄ alkyl and C₁-C₄ haloalkyl;
R₃ and R₆ are the same or different, and are each independently selected from hydrogen, halogen, —CN, C₁-C₄ alkyl, —O—C₁-C₄ alkyl, C₃-C₆ cycloalkyl and 3-6 membered heterocyclyl; the Cr C₄ alkyl, C₃-C₆ cycloalkyl and 3-6 membered heterocyclyl are optionally substituted with one or more of hydrogen, halogen, oxo, —NO₂, —OH, —NH₂, —CN, C₁-C₄ alkyl, —O—C₁-C₄ alkyl, —NH(C₁-C₄ alkyl) or —N(C₁-C₄ alkyl)₂;
R₇ is selected from halogen, —OR₉, C₁-C₄ alkyl, C₁-C₄ haloalkyl or —N(R₉)₂;
R₈ is selected from halogen, —NO₂, —CN, C₁-C₄ alkyl, C₂-C₄ alkenyl, —OR₉ or —N(R₉)₂, wherein the C₁-C₄ alkyl and C₂-C₄ alkenyl are optionally substituted with one or more of hydrogen, halogen, oxo, —NO₂, —OH, —NH₂ and —CN; and
each R₉ is the same or different, and is independently selected from hydrogen and C₁-C₄ alkyl, wherein the C₁-C₄ alkyl is optionally substituted with one or more of hydrogen, halogen, oxo, —NO₂, —OH, —NH₂ and —CN.

In some embodiments, the compound represented by formula (I), or the pharmaceutically acceptable salt, solvate, chelate, non-covalent complex or prodrug thereof is selected from a compound having a structure represented by formula (III):

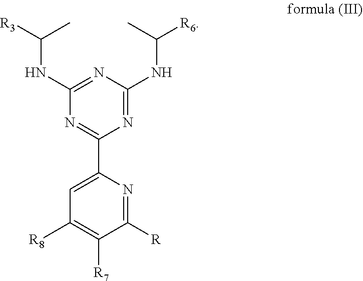

In some embodiments, the compound represented by formula (I), or the pharmaceutically acceptable salt, solvate, chelate, non-covalent complex or prodrug thereof is selected from a compound having a structure represented by formula (IV):

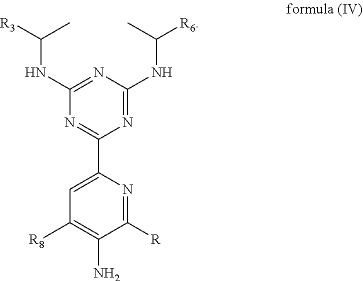

In some embodiments, the compound represented by formula (I), or the pharmaceutically acceptable salt, solvate, chelate, non-covalent complex or prodrug thereof is selected from a compound having a structure represented by formula (V):

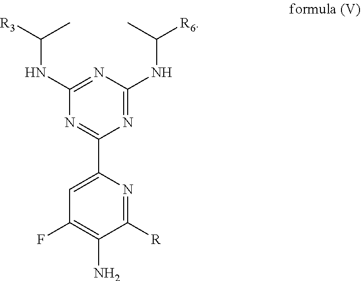

In some embodiments, the compound represented by formula (I), or the pharmaceutically acceptable salt, solvate, chelate, non-covalent complex or prodrug thereof is selected from a compound having a structure represented by formula (IIa), formula (IIIa), formula (IVa) or formula (Va):

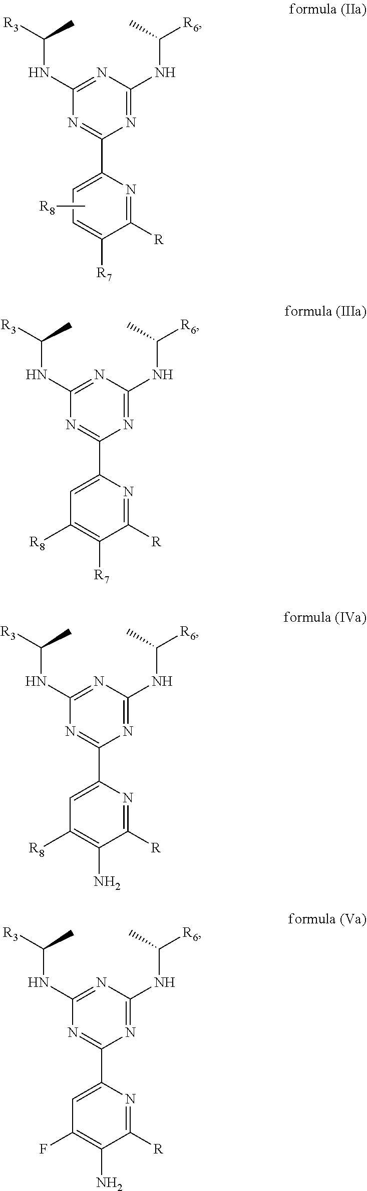

In some embodiments, the compound represented by formula (I), or the pharmaceutically acceptable salt, solvate, chelate, non-covalent complex or prodrug thereof is selected from a compound having a structure represented by formula (VI):

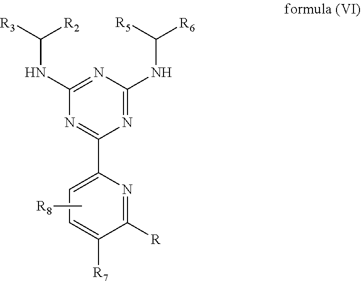

wherein
R is selected from halogen, —NO₂, —CN, —NH₂, C₁-C₄ alkyl and C₁-C₄ haloalkyl;
R₂ and R₃ optionally together with the carbon atom to which they are attached form C₃-C₁₀ cycloalkyl and 3-10 membered heterocyclyl, wherein the C₃-C₁₀ cycloalkyl and 3-10 membered heterocyclyl are optionally substituted with one or more of —OH, —NH₂, —CN, oxo, —NO₂, halogen, C₁-C₄ alkyl and C₁-C₄ alkoxy; or
R₅ and R₆ optionally together with the carbon atom to which they are attached form C₃-C₁₀ cycloalkyl and 3-10 membered heterocyclyl, wherein the C₃-C₁₀ cycloalkyl and 3-10 membered heterocyclyl are optionally substituted with one or more of —OH, —NH₂, —CN, oxo, —NO₂, halogen, C₁-C₄ alkyl and C₁-C₄ alkoxy;
R₇ is selected from halogen, —OR₉, C₁-C₄ alkyl, C₁-C₄ haloalkyl or —N(R₉)₂;
R₈ is selected from hydrogen, halogen, —NO₂, —CN, C₁-C₄ alkyl, C₂-C₄ alkenyl, —OR₉ or —N(R₉)₂, wherein the C₁-C₄ alkyl and C₂-C₄ alkenyl are optionally substituted with one or more of hydrogen, halogen, oxo, —NO₂, —OH, —NH₂ and —CN; and
each R₉ is the same or different, and is independently selected from hydrogen and C₁-C₄ alkyl, wherein the C₁-C₄ alkyl is optionally substituted with one or more of hydrogen, halogen, oxo, —NO₂, —OH, —NH₂ and —CN.

In some embodiments, the compound represented by formula (I), or the pharmaceutically acceptable salt, solvate, chelate, non-covalent complex or prodrug thereof is selected from a compound having a structure represented by formula (VIa) or (VIb):

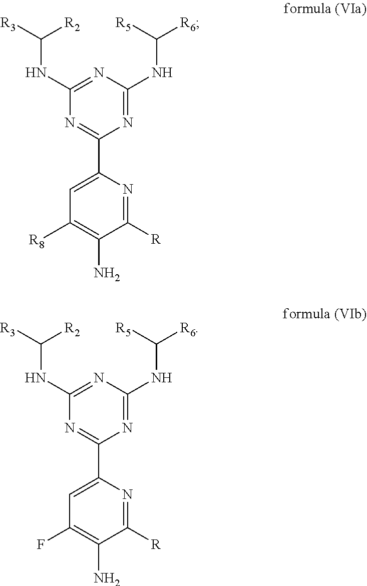

For the definitions of the groups in the above-mentioned general formula structures of the present invention, when there is no conflict in the definitions of the groups, the following preferences may be further made.

In some embodiments, R is selected from halogen and C₁-C₄ haloalkyl; further, R is selected from F, Cl, Br and CF₃.

In some embodiments, R is selected from Cl.

In some embodiments, R is selected from CF₃.

In some embodiments, R₂, R₃, R₅ and R₆ are independently selected from C₁-C₄ alkyl or C₃-C₆ cycloalkyl, wherein the C₁-C₄ alkyl and C₃-C₆ cycloalkyl are optionally substituted with one or more of hydrogen, halogen, —OH, —NH₂, —CN, C₁-C₄ alkyl, —O—C₁-C₄ alkyl, —NH(C₁-C₄ alkyl) or —N(C₁-C₄ alkyl)₂.

In some embodiments, R₂, R₃, R₅ and R₆ are independently selected from CF₃, CH₃ or cyclopropyl; further, R₂ is different from R₃, and R₅ is different from R₆.

In some embodiments, R₂ and R₅ are each independently selected from CH₃.

In some embodiments, R₃ and R₆ are each independently selected from CF₃ or cyclopropyl.

In some embodiments, R₁ and R₄ are independently selected from hydrogen.

In some embodiments, R₂ and R₃ together with the carbon atom to which they are attached form C₃-C₆ cycloalkyl, preferably cyclopropane and cyclobutane; and the C₃-C₆ cycloalkyl (cyclopropane or cyclobutane) is optionally substituted with one or more of hydrogen and halogen.

In some embodiments, R₅ and R₆ together with the carbon atom to which they are attached form C₃-C₆ cycloalkyl, preferably cyclopropane and cyclobutane; and the C₃-C₆ cycloalkyl (cyclopropane or cyclobutane) is optionally substituted with one or more of hydrogen and halogen.

In some embodiments, R₁, R₂, R₃, R₄, R₅ and R₆ are the same or different, and are each independently selected from hydrogen, —CN, —CH₃, —CH₂CH₂CH₃, —CH₂CH₂CH₂CH₃, —CHF₂, —CF₃, —CF₂CH₃, —CH₂CF₃,

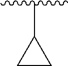 [SMILES: *C1CC1]

and —CH₂OCH₃.

In some embodiments,

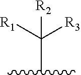

is selected from

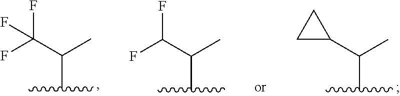

further,

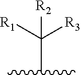

is selected from

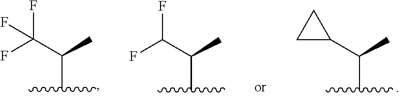

In some embodiments,

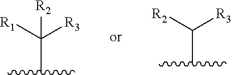

is selected from

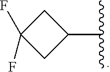 [SMILES: *C1CC(F)(F)C1]

In some embodiments,

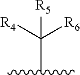

is selected from

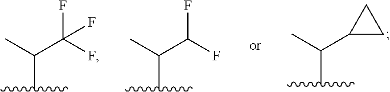

further,

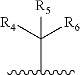

is selected from

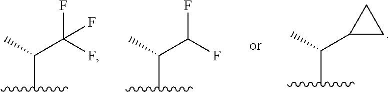

In some embodiments,

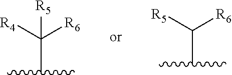

is selected from

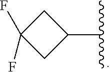 [SMILES: *C1CC(F)(F)C1]

In some embodiments, R₇ is selected from —N(R₉)₂; further, R₇ is selected from —NH₂.

In some embodiments, R₈ is selected from halogen, C₁-C₄ alkyl, C₂-C₄ alkenyl or —OR₉, wherein the C₁-C₄ alkyl and C₂-C₄ alkenyl are optionally substituted with one or more of hydrogen, halogen, —OH, —NH₂ and —CN.

In some embodiments, R₈ is selected from halogen or C₁-C₄ alkyl, wherein the C₁-C₄ alkyl is optionally substituted with one or more of hydrogen and halogen.

In some embodiments, R₈ is selected from F, Cl, Br, CH₃, CHF₂, CF₃, —OCH₃, —OCHF₂ and ethenyl.

The present invention further provides a compound represented by formula (I) or a pharmaceutically acceptable salt thereof, wherein the compound represented by formula (I) is selected from:
(6-(5-amino-4,6-dichloropyridin-2-yl)-N²,N⁴-bis(1,1,1-trifluoroprop-2-yl)-1,3,5-triazine-2,4-diamine;
(6-(5-amino-6-chloropyridin-2-yl)-N²,N⁴-bis(1,1,1-trifluoroprop-2-yl)-1,3,5-triazine-2,4-diamine;
6-(5-amino-6-chloro-4-methoxypyridin-2-yl)-N²,N⁴-bis(1,1,1-trifluoroprop-2-yl)-1,3,5-triazine-2,4-diamine;
6-(5-amino-6-chloro-3-fluoropyridin-2-yl)-N²,N⁴-bis(1,1,1-trifluoroprop-2-yl)-1,3,5-triazine-2,4-diamine;
6-(5-amino-6-chloro-4-fluoropyridin-2-yl)-N²,N⁴-bis(1,1,1-trifluoropropyl-2-yl)-1,3,5-triazine-2,4-diamine;
6-(5-amino-4-bromo-6-chloropyridin-2-yl)-N²,N⁴-bis(1,1,1-trifluoropropyl-2-yl)-1,3,5-triazine-2,4-diamine;
6-(5-amino-6-chloro-4-methylpyridin-2-yl)-N²,N⁴-bis(1,1,1-trifluoropropyl-2-yl)-1,3,5-triazine-2,4-diamine;
6-(5-amino-6-chloro-4-(difluoromethyl)pyridin-2-yl)-N²,N⁴-bis(1,1,1-trifluoropropyl-2-yl)-1,3,5-triazine-2,4-diamine;
6-(5-amino-6-chloro-4-(difluoromethoxy)pyridin-2-yl)-N²,N⁴-bis(1,1,1-trifluoropropyl-2-yl)-1,3,5-triazine-2,4-diamine;
6-(5-amino-6-chloro-4-vinylpyridin-2-yl)-N²,N⁴-bis(1,1,1-trifluoropropyl-2-yl)-1,3,5-triazine-2,4-diamine;
6-(5-amino-6-chloro-4-fluoropyridin-2-yl)-N²,N⁴-bis(1-cyclopropylethyl)-1,3,5-triazine-2,4-diamine;
6-(5-amino-6-chloro-4-fluoropyridin-2-yl)-N²,N⁴-bis(3,3-difluorocyclobutyl)-1,3,5-triazine-2,4-diamine;
6-(5-amino-4-fluoro-6-(trifluoromethyl)pyridin-2-yl)-N²,N⁴-bis(1,1,1-trifluoroprop-2-yl)-1,3,5-triazine-2,4-diamine;
6-(5-amino-4-fluoro-6-(trifluoromethyl)pyridin-2-yl)-N²,N⁴-bis(1-cyclopropylethyl)-1,3,5-triazine-2,4-diamine;
6-(5-amino-4-fluoro-6-(trifluoromethyl)pyridin-2-yl)-N²,N⁴-bis(3,3-difluorocyclobutyl)-1,3,5-triazine-2,4-diamine;
6-(5-amino-6-chloro-4-fluoropyridin-2-yl)-N²,N⁴-bis(1,1-difluoroprop-2-yl)-1,3,5-triazine-2,4-diamine;
6-(5,6-dichloro-4-fluoropyridin-2-yl)-N²,N⁴-bis(1,1,1-trifluoroprop-2-yl)-1,3,5-triazine-2,4-diamine;
6-(5-bromo-6-chloro-4-fluoropyridin-2-yl)-N²,N⁴-bis(1,1,1-trifluoroprop-2-yl)-1,3,5-triazine-2,4-diamine;
6-(4,6-dichloro-5hydroxylpyridin-2-yl)-N²,N⁴-bis(1,1,1-trifluoroprop-2-yl)-1,3,5-triazine-2,4-diamine;
6-(4,6-dichloro-5-methylamino pyridin-2-yl)-N²,N⁴-bis(1,1,1-trifluoroprop-2-yl)-1,3,5-triazine-2,4-diamine;
6-(4,6-dichloro-5-dimethylamino pyridin-2-yl)-N²,N⁴-bis(1,1,1-trifluoroprop-2-yl)-1,3,5-triazine-2,4-diamine;
6-(4,6-bis((1,1,1-trifluoroprop-2-yl)amine)-1,3,5-triazin-2-yl)-2-chloro-4-fluoropyridin-3-ol;
6-(5-amino-4,6-difluoropyridin-2-yl)-N²,N⁴-bis(1,1,1-trifluoroprop-2-yl)-1,3,5-triazine-2,4-diamine;
6-(6-chloro-4-fluoro-5-aminopyridin-2-yl)-N²,N⁴-dicyclopropyl-1,3,5-triazine-2,4-diamine;
6-(6-chloro-5-amino-4-(trifluoromethyl)pyridin-2-yl)-N²,N⁴-bis(1,1,1-trifluoropropyl-2-yl)-1,3,5-triazine-2,4-diamine;
6-(5-amino-6-chloro-4-fluoropyridin-2-yl)-N²,N⁴-diisopropyl-1,3,5-triazine-2,4-diamine;
6-(5-amino-6-chloro-3-fluoropyridin-2-yl)-N²,N⁴-bis(1-cyclopropylethyl)-1,3,5-triazine-2,4-diamine;
6-(5-amino-6-chloro-3-fluoropyridin-2-yl)-N²,N⁴-bis(1,1-difluoroprop-2-yl)-1,3,5-triazine-2,4-diamine;
6-(5-amino-6-chloro-3-fluoropyridin-2-yl)-N²,N⁴-diisopropyl-1,3,5-triazine-2,4-diamine;
6-(5-amino-6-chloro-3-fluoropyridin-2-yl)-N²,N⁴-dicyclopropyl-1,3,5-triazine-2,4-diamine;
6-(5-amino-6-chloro-3-fluoropyridin-2-yl)-N²,N⁴-dicyclobutyl-1,3,5-triazine-2,4-diamine; or
6-(5-amino-6-chloro-3-fluoropyridin-2-yl)-N²,N⁴-bis(3,3-difluorocyclobutyl-1,3,5-triazine-2,4-diamine.

The present invention further provides a compound represented by formula (I) or a pharmaceutically acceptable salt thereof, wherein the compound represented by formula (I) is selected from:
(6-(5-amino-4,6-dichloropyridin-2-yl)-N²,N⁴-bis((R)-1,1,1-trifluoroprop-2-yl)-1,3,5-triazine-2,4-diamine;
(6-(5-amino-6-chloropyridin-2-yl)-N²,N⁴-bis((R)-1,1,1-trifluoroprop-2-yl)-1,3,5-triazine-2,4-diamine;
6-(5-amino-6-chloro-4-methoxypyridin-2-yl)-N²,N⁴-bis((R)-1,1,1-trifluoroprop-2-yl)-1,3,5-triazine-2,4-diamine;
6-(5-amino-6-chloro-3-fluoropyridin-2-yl)-N²,N⁴-bis((R)-1,1,1-trifluoroprop-2-yl)-1,3,5-triazine-2,4-diamine;
6-(5-amino-6-chloro-4-fluoropyridin-2-yl)-N²,N⁴-bis((R)-1,1,1-trifluoropropyl-2-yl)-1,3,5-triazine-2,4-diamine;
6-(5-amino-4-bromo-6-chloropyridin-2-yl)-N²,N⁴-bis((R)-1,1,1-trifluoropropyl-2-yl)-1,3,5-triazine-2,4-diamine;
6-(5-amino-6-chloro-4-methylpyridin-2-yl)-N²,N⁴-bis((R)-1,1,1-trifluoropropyl-2-yl)-1,3,5-triazine-2,4-diamine;
6-(5-amino-6-chloro-4-(difluoromethyl)pyridin-2-yl)-N²,N⁴-bis((R)-1,1,1-trifluoropropyl-2-yl)-1,3,5-triazine-2,4-diamine;
6-(5-amino-6-chloro-4-(difluoromethoxy)pyridin-2-yl)-N²,N⁴-bis((R)-1,1,1-trifluoropropyl-2-yl)-1,3,5-triazine-2,4-diamine;
6-(5-amino-6-chloro-4-vinylpyridin-2-yl)-N²,N⁴-bis((R)-1,1,1-trifluoropropyl-2-yl)-1,3,5-triazine-2,4-diamine;
6-(5-amino-6-chloro-4-fluoropyridin-2-yl)-N²,N⁴-bis((R)-1-cyclopropylethyl)-1,3,5-triazine-2,4-diamine;
6-(5-amino-6-chloro-4-fluoropyridin-2-yl)-N²,N⁴-bis(3,3-difluorocycIobutyl)-1,3,5-triazine-2,4-diamine;
6-(5-amino-4-fluoro-6-(trifluoromethyl)pyridin-2-yl)-N²,N⁴-bis((R)-1,1,1-trifluoroprop-2-yl)-1,3,5-triazine-2,4-diamine;
6-(5-amino-4-fluoro-6-(trifluoromethyl)pyridin-2-yl)-N²,N⁴-bis((R)-1-cyclopropylethyl)-1,3,5-triazine-2,4-diamine;
6-(5-amino-4-fluoro-6-(trifluoromethyl)pyridin-2-yl)-N²,N⁴-bis(3,3-difluorocyclobutyl)-1,3,5-triazine-2,4-diamine;
6-(5-amino-6-chloro-4-fluoropyridin-2-yl)-N²,N⁴-bis((R)-1,1-difluoroprop-2-yl)-1,3,5-triazine-2,4-diamine;
6-(5,6-dichloro-4-fluoropyridin-2-yl)-N²,N⁴-bis((R)-1,1,1-trifluoroprop-2-yl)-1,3,5-triazine-2,4-diamine;
6-(5-bromo-6-chloro-4-fluoropyridin-2-yl)-N²,N⁴-bis((R)-1,1,1-trifluoroprop-2-yl)-1,3,5-triazine-2,4-diamine;
6-(4,6-dichloro-5hydroxylpyridin-2-yl)-N²,N⁴-bis((R)-1,1,1-trifluoroprop-2-yl)-1,3,5-triazine-2,4-diamine;
6-(4,6-dichloro-5-methylamino pyridin-2-yl)-N,N*-bis((R)-1,1,1-trifluoroprop-2-yl)-1,3,5-triazine-2,4-diamine;
6-(4,6-dichloro-5-dimethylamino pyridin-2-yi)-N²,N⁴_bis((R)-1,1,1-trifluoroprop-2-yl)-1,3,5-triazine-2,4-diamine;
6-(4,6-bis(((R)-1,1,1-trifluoroprop-2-yl)amine)-1,3,5-triazin-2-yl)-2-chloro-4-fluoropyridin-3-ol;
6-(5-amino-4,6-difluoropyridin-2-yl)-N²,N⁴_bis((R)-1,1,1-trifluoroprop-2-yl)-1,3,5-triazine-2,4-diamine;
6-(6-chloro-4-fluoro-5-aminopyridin-2-yi)-N²,N⁴-dicyclopropyl-1,3,5-triazine-2,4-diamine;
6-(6-chloro-5-amino-4-(trifluoromethyl)pyridin-2-yl)-N²,N⁴-bis((R)-1,1,1-trifluoropropyl-2-yl)-1,3,5-triazine-2,4-diamine;
6-(5-amino-6-chloro-4-fluoropyridin-2-yl)-N²,N⁴-diisopropyl-1,3,5-triazine-2,4-diamine;
6-(5-amino-6-chloro-3-fluoropyridin-2-yl)-N²,N⁴-bis((R)-1-cyclopropylethyl)-1,3,5-triazine-2,4-diamine;
6-(5-amino-6-chloro-3-fluoropyridin-2-yl)-N²,N⁴-bis(1,1-difluoroprop-2-yl)-1,3,5-triazine-2,4-diamine;
6-(5-amino-6-chloro-3-fluoropyridin-2-yl)-N²,N⁴-diisopropyl-1,3,5-triazine-2,4-diamine;
6-(5-amino-6-chloro-3-fluoropyridin-2-yl)-N²,N⁴-dicyclopropyl-1,3,5-triazine-2,4-diamine;
6-(5-amino-6-chloro-3-fluoropyridin-2-yl)-N²,N⁴-dicyclobutyl-1,3,5-triazine-2,4-diamine;
6-(5-amino-6-chloro-3-fluoropyridin-2-yl)-N²,N⁴-bis(3,3-difluorocyclobutyl-1,3,5-triazine-2,4-diamine.

The present invention also provides a pharmaceutical composition comprising a therapeutically effective amount of at least one of the above-mentioned compounds and at least one pharmaceutically acceptable excipient.

The present invention provides the use of the compound represented by formula (I) or the pharmaceutical composition in the preparation of a drug.

The present invention further provides a preferred technical solution of the use.

Preferably, the use is to treat, prevent, delay or halt the occurrence or progression of cancer or cancer metastasis.

Preferably, the use is for the treatment of a mutant IDH1 and IDH2-mediated disease.

Preferably, the disease is cancer.

Preferably, the cancer is selected from brain glioma, melanoma, papillary thyroid tumor, cholangiocarcinoma, lung cancer, breast cancer, sarcoma, glioma, glioblastoma multiforme, acute myeloid leukemia, non-Hodgkin's lymphoma, etc. In specific embodiments, the cancer to be treated is brain glioma, glioblastoma (glioma), myelodysplastic syndrome (MDS), myeloproliferative neoplasms (MPN), acute myeloid leukemia (AML), sarcoma, melanoma, non-small cell lung cancer, chondrosarcoma, cholangiocarcinoma or angioimmunoblastic lymphoma. In more specific embodiments, the cancer to be treated is brain glioma, glioblastoma (glioma), myelodysplastic syndrome (MDS), myeloproliferative neoplasms (MPN), acute myeloid leukemia (AML), melanoma, chondrosarcoma or angioimmunoblastic non-Hodgkin's lymphoma (NHL).

Preferably, the use is for use as a mutant IDH1 and IDH2 inhibitor.

The present invention further provides a method for treating and/or preventing an IDH1 and IDH2-mediated disease by administering a therapeutically effective amount of at least any one of a compound represented by formula (I) or a pharmaceutical composition to a subject to be treated.

Preferably, in the above-mentioned method, the IDH1 and IDH2-mediated disease is cancer.

The present invention further provides a method for treating cancer, wherein the method comprises administering a therapeutically effective amount of at least any one of a compound represented by formula (I) or a pharmaceutical composition to a subject to be treated. In some embodiments, the present invention relates to a method for treating cancer characterized by the presence of mutant IDH1 and IDH2, comprising administering a therapeutically effective amount of at least any one of a compound represented by formula (I) or an isomer, a pharmaceutically acceptable salt, crystal, solvate or prodrug thereof, or a pharmaceutical composition comprising same to a patient in need thereof, wherein the cancer is selected from brain glioma, melanoma, papillary thyroid tumor, cholangiocarcinoma, lung cancer, breast cancer, sarcoma, glioma, glioblastoma multiforme, acute myeloid leukemia, non-Hodgkin's lymphoma, etc.

Preferably, in the above-mentioned method, the subject to be treated is human.

### Definitions

The general chemical terms used in the above-mentioned structural general formulas have common meanings.

For example, the terms “halo” and “halogen” as used herein refer to fluorine, chlorine, bromine or iodine, unless otherwise indicated. The preferred halogen groups include fluorine, chlorine and bromine.

In the present invention, unless otherwise indicated, “alkyl” includes a linear or branched monovalent saturated hydrocarbon group. As used herein, the alkyl groups may be optionally substituted with one to more substituents. Non-limiting examples of alkyl groups include, for example, methyl, ethyl, propyl, isopropyl, n-butyl, isobutyl, sec-butyl, tert-butyl, n-pentyl, 3-(2-methyl)butyl, 2-pentyl, 2-methylbutyl, neopentyl, n-hexyl, 2-hexyl and 2-methylpentyl. Similarly, the “C₁₋₄” in the “C₁₋₄ alkyl” refers to a group containing 1, 2, 3 or 4 carbon atoms arranged in the form of a linear or branched chain.

Alkenyl and alkynyl include linear or branched alkenyl and alkynyl. Similarly, “C₂₋₄ alkenyl” and “C₂₋₄ alkynyl” refer to alkenyl or alkynyl containing 2, 3 or 4 carbon atoms arranged in the form of a linear or branched chain.

“Haloalkyl” means that the aforementioned linear or branched alkyl is substituted with one or more halogens. Non-limiting examples of haloalkyl include, but are not limited to, for example, —CH₂F, —CHF₂, —CF₃, —CH₂CH₂F, —CH₂CHF₂, —CH₂CF₃, —CHFCH₃, —CF₂CH₃ and —CHFCH₂F.

“Alkoxy” refers to oxygen ether forms of the aforementioned linear or branched alkyl, i.e., —O-alkyl.

In the present invention, “a”, “an”, “the”, “at least one”, and “one or more” are used interchangeably. Therefore, for example, a composition comprising “a” pharmaceutically acceptable excipient can be interpreted to mean that the composition includes “one or more” pharmaceutically acceptable excipients.

Unless otherwise indicated, the term “aromatic ring” in the present invention refers to unsubstituted or substituted monocyclic, bicyclic or fused-ring aromatic groups containing carbon atoms, or unsubstituted or substituted monocyclic, bicyclic or fused-ring aromatic groups containing heteroatoms such as N, O or S, and when an aromatic ring is bicyclic or fused-ring, at least one of the rings is aromatic. The aromatic ring is preferably a 5 to 10 membered monocyclic or bicyclic aromatic ring group. Examples of the aromatic rings include, but are not limited to phenyl, pyridyl, pyrazolyl, pyrimidyl, benzodihydrofuran and indolyl.

The term “cycloalkyl” refers to monocyclic and polycyclic ring systems containing only carbon atoms in the ring and may be optionally substituted with one to more substituents. As used herein, cycloalkyl refers to and includes a saturated or unsaturated non-aromatic ring system. The term cycloalkyl further includes bridged, fused and spirocyclic ring systems. Non-limiting examples of cycloalkyl include, for example, cyclopropyl, cyclobutyl, cyclopentyl, cyclohexyl, cyclohexenyl, spiro[3.4]octyl and bicyclic[2.2.1]heptane.

Unless otherwise indicated, the term “heterocyclyl” in the present invention refers to unsubstituted or substituted monocyclic and polycyclic ring systems consisting of carbon atoms and 1-3 heteroatoms selected from N, O or S, and includes saturated or unsaturated ring systems and polycyclic ring systems with unsaturated moieties and/or aromatic moieties. The nitrogen or sulfur heteroatoms may be optionally oxidized, and the nitrogen heteroatom may be optionally quaternized. The heterocyclyl may be attached at any heteroatom or carbon atom to form a stable structure. It should be understood that polycyclic heterocycloalkyl groups further include fused, bridged and spirocyclic ring systems. As used herein, a heterocycloalkyl group may be optionally substituted with one to more substituents. Examples of the heterocyclyl include, but are not limited to azetidinyl, pyrrolidyl, piperidyl, piperazinyl, oxopiperazinyl, oxopiperidyl, tetrahydrofuryl, dioxolanyl, tetrahydroimidazolyl, tetrahydrothiazolyl, tetrahydrooxazolyl, tetrahydropyranyl, morpholinyl, thiomorpholinyl, thiomorpholinyl sulfoxide, thiomorpholinyl sulfone and tetrahydrooxadiazolyl.

Unless otherwise indicated, the term “aryl” as used herein refers to an unsubstituted or substituted monocyclic or polycyclic ring system containing carbon ring atoms, and at least one of the rings is aromatic. The preferred aryls are monocyclic or bicyclic 6-10 membered aromatic ring systems. Phenyl and naphthyl are preferred aryls. The most preferred aryl is phenyl.

Unless otherwise indicated, the term “heteroaryl” in the present invention refers to an unsubstituted or substituted stable 5 or 6 membered monocyclic aromatic ring system or an unsubstituted or substituted 9 or 10 membered benzo-fused heteroaromatic ring system or bicyclic heteroaromatic ring system, which consists of carbon atoms and 1-4 heteroatoms selected from N, O or S, wherein the nitrogen or sulfur heteroatoms may be optionally oxidized, and the nitrogen heteroatom may be optionally quaternized. The heteroaryl may be attached at any heteroatom or carbon atom to form a stable structure. Examples of heteroaryl include, but are not limited to thienyl, furyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuryl, benzothienyl, benzisoxazolyl, benzothiazolyl, benzothiazolyl, benzothiadiazolyl, benzotriazolyl adeninyl, quinolyl or isoquinolyl.

The term “substituted” means that one or more hydrogen atoms in a group are each replaced by the same or different substituent(s). Typical substituents include, but are not limited to, halogen (F, Cl, Br or I), C₁₋₈ alkyl, C₃₋₁₂ cycloalkyl, —OR¹, —SR¹, ═O, ═S, —C(O)R¹, —C(S)R¹, =NR¹, —C(O)OR¹, —C(S)OR¹, —NR¹R², —C(O)NR¹R², cyano, nitro, —S(O)₂R¹, —O—S(O₂)OR¹, —O—S(O)₂R¹ and —OP(O)(OR¹)(OR²), wherein R¹ and R² are independently selected from —H, C₁₋₆ alkyl and C₁₋₆ haloalkyl. In some embodiments, substituents are independently selected from the groups comprising —F, —Cl, —Br, —I, —OH, trifluoromethoxy, ethoxy, propoxy, isopropoxy, n-butoxy, isobutoxy, tert-butoxy, —SCH₃, —SC₂H₅, formaldehyde, —C(OCH₃), cyano, nitro, —CF₃, —OCF₃, amino, dimethylamino, methylthio, sulfonyl and acetyl.

Examples of substituted alkyl include, but are not limited to, 2-aminoethyl, 2-hydroxyethyl, pentachloroethyl, trifluoromethyl, methoxymethyl, pentafluoroethyl and piperazinylmethyl.

Examples of substituted alkoxy include, but are not limited to, aminomethoxy, trifluoromethoxy, 2-diethylaminoethoxy, 2-ethoxycarbonylethoxy and 3-hydroxypropoxy.

The term “pharmaceutically acceptable salt” refers to a salt prepared from a pharmaceutically acceptable non-toxic base or acid. When the compound provided by the present invention is an acid, its corresponding salt can be conveniently prepared from pharmaceutically acceptable non-toxic bases, including inorganic bases and organic bases. Salts derived from inorganic bases include aluminum, ammonium, calcium, copper (supervalent and subvalent), ferric, ferrous, lithium, magnesium, manganese (supervalent and subvalent), potassium, sodium, zinc and the like. Particularly preferred are the ammonium, calcium, magnesium, potassium and sodium salts. Pharmaceutically acceptable non-toxic organic bases from which salts can be derived include primary, secondary, and tertiary amines, including cyclic amines and substituent-containing amines, such as naturally occurring and synthetic substituent-containing amines. Other pharmaceutically acceptable non-toxic organic bases capable of forming salts include ion exchange resins and arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, reduced glucosamine, glucosamine, histidine, hydrabamine, isopropylamine, lysine, methylglucamine, morpholine, piperazine, piperidine, polyamine resins, procaine, purine, theobromine, triethylamine, trimethylamine, tripropylamine, tromethamine, and the like.

When the compound provided by the present invention is a base, its corresponding salt can be conveniently prepared from pharmaceutically acceptable non-toxic acids, including inorganic acids and organic acids. Such acids include, for example, acetic acid, benzenesulfonic acid, benzoic acid, camphorsulfonic acid, citric acid, ethanesulfonic acid, ethylenhydrin-sulfonic acid, formic acid, fumaric acid, gluconic acid, glutamic acid, hydrobromic acid, hydroiodic acid, perchloric acid, hydrochloric acid, isethionic acid, propanoic acid, glycolic acid, lactic acid, maleic acid, malic acid, mandelic acid, methanesulfonic acid, mucic acid, nitric acid, oxalic acid, pamoic acid, pantothenic acid, phosphoric acid, succinic acid, sulfuric acid, 2-naphthalenesulfonic acid, cyclohexylamine sulfonic acid, salicylic acid, saccharinic acid, trifluoroacetic acid, tartaric acid, p-toluenesulfonic acid, etc.; preferably, citric acid, hydrobromic acid, formic acid, hydrochloric acid, maleic acid, phosphoric acid, sulfuric acid and tartaric acid; more preferably, formic acid and hydrochloric acid.

Since the compound represented by formula (I) will be used as a drug, the compound is preferably used at a certain purity, for example, at least 60% purity, more suitably at least 75% purity, and particularly suitably at least 98% purity (% represents weight ratio).

The prodrugs of the compounds of the present invention are included in the scope of protection of the present invention. Generally, the prodrug refers to a functional derivative that is readily converted into the desired compound in vivo. For example, after administered to a subject, any pharmaceutically acceptable salt, ester, salt of an ester or other derivatives of the compound of the present application can directly or indirectly provide the compound of the present application or a pharmaceutically active metabolite or residue thereof. Particularly preferred derivatives or prodrugs are the compounds which, when administered to patients, can improve the bioavailability of the compounds of the present application (for example, can make an orally administered compound more easily absorbed into the blood), or promote the delivery of parent compounds to biological organs or sites of action (for example, the brain or lymphatic system). Therefore, the term “administering” in the treatment methods provided by the present invention refers to the administration of the compounds disclosed in the present invention that can treat different diseases, or the compounds which are not explicitly disclosed, but can be converted into the compounds disclosed in the present invention in vivo after administration to a subject. Conventional methods for selecting and preparing suitable prodrug derivatives are described in books, e.g., Design of Prodrugs, ed. H. Bundgaard, Elsevier, 1985.

Obviously, the definition of any substituent or a variable at a particular position in a molecule is independent of its definitions at other positions in the molecule. It is easy to understand that those skilled in the art can select substituents or substitution forms of the compounds of the present invention by means of existing technical means and the methods described in the present invention so as to obtain chemically stable and easily synthesized compounds.

The compounds of the present invention may contain one or more asymmetric centers and therefore may give rise to diastereomers and optical isomers. The present invention includes all possible diastereomers and racemic mixtures thereof, substantially pure resolved enantiomers thereof, all possible geometric isomers and pharmaceutically acceptable salts thereof.

The present invention includes all stereoisomers of the compounds represented by formula (I) and pharmaceutically acceptable salts thereof. Further, mixtures of stereoisomers as well as isolated specific stereoisomers are also included in the present invention. During the synthetic process for preparing such compounds, or during racemization or epimerization known to those skilled in the art, the products obtained thereby may be mixtures of stereoisomers.

When a tautomer of the compound represented by formula (I) exists, the present invention includes any possible tautomer and pharmaceutically acceptable salt thereof, and mixtures thereof, unless otherwise specifically stated.

When a solvate or polymorph of the compound represented by formula (I) and the pharmaceutically acceptable salt thereof exists, the present invention includes any possible solvates and polymorphs. The type of solvent forming the solvate is not particularly limited as long as the solvent is pharmaceutically acceptable. For example, solvents, such as water, ethanol, propanol, and acetone, may be used.

The term “composition” in the present invention is intended to include a product comprising specified ingredients in specified amounts, as well as any product which is produced, directly or indirectly, from combinations of the specified ingredients in the specified amounts. Accordingly, pharmaceutical compositions containing the compounds of the present invention as active ingredients and methods for preparing the compounds of the present invention are also part of the present invention. Furthermore, some of the crystalline forms for the compounds may exist as polymorphs, and such polymorphs are included in the present invention. In addition, some of the compounds may form solvates with water (i. e., hydrates) or common organic solvents, and such solvates also fall within the scope of the present invention.

The pharmaceutical composition provided by the present invention comprises a compound represented by formula (I) (or a pharmaceutically acceptable salt thereof) as an active component, a pharmaceutically acceptable excipient and other optional therapeutic components or excipients. Although the most suitable mode of administration of the active component in any given case depends on the specific subject to be administered, the nature of the subject and the severity of the condition, the pharmaceutical compositions of the present invention include those suitable for oral, rectal, topical and parenteral (including subcutaneous administration, intramuscular injection, and intravenous administration) administration. The pharmaceutical compositions of the present invention may be conveniently presented in unit dosage form well known in the art and prepared by any methods well known in the art of pharmacology.

In fact, according to conventional drug mixing techniques, the compound represented by formula (I) of the present invention, or a prodrug, or a metabolite, or a pharmaceutically acceptable salt, can be used as an active component and mixed with a drug carrier to form a pharmaceutical composition. The drug carrier may take a wide variety of forms depending on the desired mode of administration, for example, oral administration or injection (including intravenous injection). Therefore, the pharmaceutical compositions of the present invention can be presented as discrete units suitable for oral administration such as capsules, cachets or tablets containing a predetermined dose of the active component. Further, the pharmaceutical composition of the present invention may be in the form of a powder, granule, solution, aqueous suspension, non-aqueous liquid, oil-in-water emulsion, or water-in-oil emulsion. Furthermore, in addition to the common dosage forms mentioned above, the compound represented by formula (I) or the pharmaceutically acceptable salt thereof can also be administered via a controlled release method and/or a delivery device. The pharmaceutical composition of the present invention can be prepared by any pharmaceutical method. In general, such methods include the step of bringing into association the active component with the carrier which constitutes one or more necessary ingredients. In general, the pharmaceutical compositions are prepared by uniformly and thoroughly admixing the active component with liquid carriers or finely divided solid carriers or a mixture of both. In addition, the product can be conveniently prepared into the desired presentation.

Therefore, the pharmaceutical composition of the present invention comprises a pharmaceutically acceptable carrier and a compound represented by formula (I), or a stereoisomer, tautomer, polymorph, solvate, pharmaceutically acceptable salt or prodrug thereof. The combined administration of the compound represented by formula (I) or the pharmaceutically acceptable salt thereof, together with one or more other therapeutically active compounds is also included in the pharmaceutical composition of the present invention.

The drug carrier used in the present invention may be, for example, a solid carrier, a liquid carrier or a gaseous carrier. Solid carriers include, but are not limited to lactose, gypsum powder, sucrose, talc, gelatin, agar, pectin, gum arabic, magnesium stearate and stearic acid. Liquid carriers include, but are not limited to syrup, peanut oil, olive oil and water. Gaseous carriers include, but are not limited to carbon dioxide and nitrogen. For preparing oral pharmaceutical preparations, any convenient pharmaceutical media may be employed. For example, water, glycols, oils, alcohols, flavor enhancers, preservatives, coloring agents, etc. can be used in oral liquid preparations such as suspensions, elixirs, and solutions; carriers such as starches, sugars, microcrystalline cellulose, diluents, granulating agents, lubricants, binders, disintegrants, etc. can be used in oral solid preparations such as powders, capsules and tablets. Considering the ease of administration, tablets and capsules are preferred for oral preparations, and accordingly solid pharmaceutical carriers are used. Alternatively, tablets may be coated using standard aqueous or nonaqueous preparation techniques.

Tablets containing the compounds or pharmaceutical compositions of the present invention may be prepared by compression or molding, optionally with one or more auxiliary components or adjuvants. Compressed tablets may be prepared by compressing, in a suitable machine, the active component in a free-flowing form such as powder or granules mixed with a binder, lubricant, inert diluent, surfactant or dispersing agent. Molded tablets may be made by impregnating the powdered compound or pharmaceutical composition with an inert liquid diluent and then molding same in a suitable machine.

The pharmaceutical composition suitable for parenteral administration provided by the present invention can be prepared into an aqueous solution or suspension by adding active components into water. A suitable surfactant such as hydroxypropylcellulose may be included. Dispersion systems can also be prepared in glycerol, liquid polyethylene glycols and mixtures thereof in oils. Further, a preservative may also be included in the pharmaceutical compositions of the present invention to prevent the growth of harmful microorganisms.

The present invention provides pharmaceutical compositions suitable for injection, including sterile aqueous solutions or dispersion systems. Further, the above pharmaceutical composition can be prepared in the form of sterile powders for the immediate preparation of sterile injections or dispersions. Regardless, the final injection form must be sterile and must be flowable for ease of injection. Furthermore, the pharmaceutical composition must be stable during manufacture and storage. Therefore, preferably, the pharmaceutical composition should be preserved against the contaminating action of microorganisms such as bacteria and fungi. The carrier can be a solvent or dispersion medium, for example, water, ethanol, polyol (e.g., glycerol, propylene glycol, liquid polyethylene glycol), vegetable oils, and suitable mixtures thereof.

The pharmaceutical composition provided by the present invention may be in a form suitable for topical administration, for example, an aerosol, emulsion, ointment, lotion, dusting powder or other similar dosage forms. Further, the pharmaceutical compositions provided by the present invention may be in a form suitable for use with a transdermal administration device. These preparations can be prepared using the compound represented by formula (I) of the present invention, or a pharmaceutically acceptable salt thereof, by conventional processing methods. As an example, by adding about 5 wt % to 10 wt % of a hydrophilic material and water to an emulsion or ointment, the emulsion or ointment having a desired consistency is prepared.

The pharmaceutical composition provided by the present invention can be in a form suitable for rectal administration with a solid as a carrier. A unit dose suppository is the most typical dosage form. Suitable excipients include cocoa butter and other materials commonly used in the art. Suppositories can be conveniently prepared by first mixing the pharmaceutical composition with softened or melted excipients, followed by cooling and molding.

In addition to the above-mentioned excipient components, the above-mentioned preparation formulas may also include, as appropriate, one or more additional excipient components, such as diluents, buffers, flavoring agents, binders, surfactants, thickeners, lubricants and preservatives (including antioxidants). Further, other adjuvants may also include penetration enhancers that adjust the osmotic pressure of the drug and blood. The pharmaceutical composition comprising the compound represented by formula (I) or a pharmaceutically acceptable salt thereof can be prepared in the form of a powder or a concentrated solution.

However, it is understood that lower or higher doses than those recited above may be required. The specific dosage level and treatment regimens for any particular patient will depend on a variety of factors, including the activity of the specific compound used, age, weight, general health, sex, diet, time of administration, route of administration, rate of excretion, concomitant drug use and the severity of the particular disease being treated.

Excellent effects of the present invention: the compounds of the present invention have excellent enzymatic and cellular activities, kinetic solubility and oral drug absorption exposures, and can be used to treat mutant IDH1 and IDH2-mediated diseases.

### BRIEF DESCRIPTION OF THE DRAWINGS

FIG. 1 shows a concentration change curve of the compounds in plasma after intravenous administration and oral gavage administration in adult beagle dogs, wherein a horizontal coordinate represents time after administration (h), and a vertical coordinate represents the concentration of the compounds in mouse plasma (ng/ml), including control groups with AG-881 and groups with the compound of example 5.

### DETAILED DESCRIPTION OF EMBODIMENTS

In order to make the above content clearer and more specific, the present invention will further illustrate the technical solution of the present invention with the following examples. The following examples are only used to illustrate specific embodiments of the present invention, so that those skilled in the art can understand the present invention, but are not used to limit the protection scope of the present invention. In the specific embodiments of the present invention, technical means or methods, etc. that are not specifically described are conventional technical means or methods, etc. in the art, and the raw materials, reagents, etc. used are all commercially available products.

Unless otherwise indicated, all fractions and percentages of the present invention are by weight, and all temperatures are in degrees Celsius.

The following abbreviations are used in the examples:
Pd(dppf)Cl₂·CH₂Cl₂: [1,1′-bis(diphenylphosphino)ferrocene]palladium dichloride dichloromethane complex;
KOAc: potassium acetate;
DMF: N,N-dimethylformamide;
DMSO: dimethyl sulfoxide;
EA: ethyl acetate;
THF: tetrahydrofuran;
1,4-dioxane: 1,4-Dioxane;
DIEA: diisopropylethylamine;
α-KG: α-ketoglutarate;
2-HG: 2-hydroxyglutaric acid;
LC-MS or LCMS: liquid chromatography-mass spectrometry.

### PREPARATION OF INTERMEDIATES

### Preparation of intermediate 6-chloro-N,N-bis[(2R)-1,1,1-trifluoropropan-2-yl]-1,3,5-triazine-2,4-diamine (Int-1)

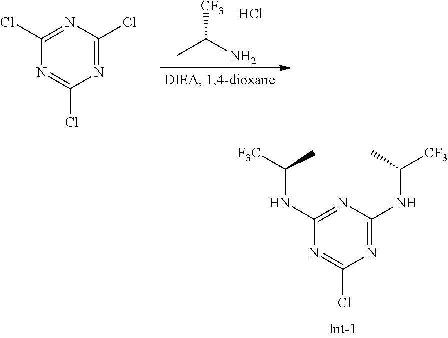

A mixture of cyanuric chloride (18.4 g, 0.1 mol), (R)-1,1,1-trifluoroisopropylamine hydrochloride (29.9 g, 0.2 mol) and 1,4-dioxane (200 mL) was cooled to 0° C., and then DIEA (100 mL, 0.6 mol) was slowly added dropwise. After the dropwise addition was completed, the reaction system was naturally warmed to room temperature and stirred for additional 30 min, and then heated to 80° C. and stirred for additional 2 h. After TLC detected that the reaction was completed, the reaction mixture was concentrated under reduced pressure, and the concentrated residue was purified by column chromatography (ethyl acetate:n-hexane=5%-10%), to obtain compound Int-1 (26.5 g) as a white to light-yellow solid. LCMS [M+H⁺] 338.05.

### Preparation of intermediate 6-chloro-N,N-bis[(R)-cyclopropan-2-yl]-1,3,5-triazine-2,4-diamine (Int-2)

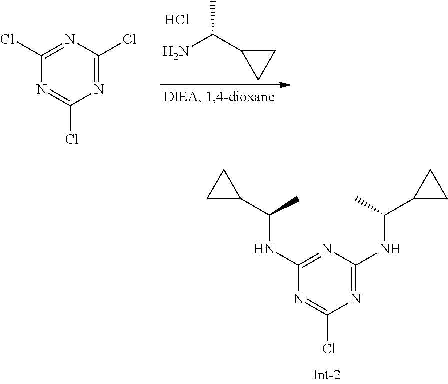

A mixture of cyanuric chloride (2.0 g, 10.85 mmol), (R)-cyclopropylethylamine hydrochloride (2.77 g, 22.78 mmol) and 1,4-dioxane (60 mL) was cooled to 0° C., and then DIEA (8.96 mL, 54.23 mmol) was slowly added dropwise. After the dropwise addition was completed, the reaction system was naturally warmed to room temperature and stirred for additional 1 h, and then heated to 60° C. and stirred for additional 1 h. After TLC detected that the reaction was completed, the reaction was cooled to room temperature. The reaction mixture was poured into water, extracted twice with ethyl acetate, washed with saturated brine, dried over anhydrous sodium sulfate and filtered by suction, and the filtrate was dried by rotary evaporation. The concentrated residue was purified by column chromatography (ethyl acetate:n-hexane=0%-20%), to obtain compound Int-2 (2.76 g) as a white to light-yellow solid. LCMS [M+H⁺] 282.14.

### Preparation of intermediate 6-chloro-N²,N⁴-bis(3,3-difluorocyclobutyl)-1,3,5-triazine-2,4-diamine (Int-3)

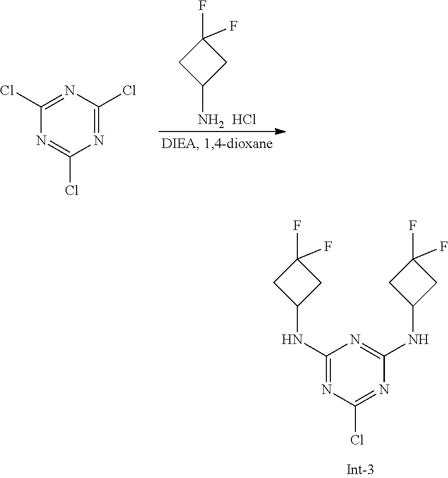 [SMILES: Clc1nc(Cl)nc(Cl)n1 ; NC1CC(F)(F)C1 ; Cl ; FC1(F)CC(Nc2nc(Cl)nc(NC3CC(F)(F)C3)n2)C1]

A mixture of cyanuric chloride (2.0 g, 10.85 mmol), 3,3-difluorocyclobutan-1-amine hydrochloride (3.1 g, 22.78 mmol) and 1,4-dioxane (60 mL) was cooled to 0° C., and then DIEA (8.96 mL, 54.23 mmol) was slowly added dropwise. After the dropwise addition was completed, the reaction system was naturally warmed to room temperature and stirred for additional 1 h, and then heated to 60° C. and stirred for additional 1 h. After TLC detected that the reaction was completed, the reaction was cooled to room temperature. The reaction mixture was poured into water, extracted twice with ethyl acetate, washed with saturated brine, dried over anhydrous sodium sulfate and filtered by suction, and the filtrate was dried by rotary evaporation. The concentrated residue was purified by column chromatography (ethyl acetate:n-hexane=0%-20%), to obtain compound Int-3 (3.2 g) as a white to light-yellow solid. LCMS [M+H⁺] 326.07

### Example 1: preparation of (6-(5-amino-4,6-dichloropyridin-2-yl)-N²,N⁴-bis((R)-1,1,1-trifluoroprop-2-yl)-1,3,5-triazine-2,4-diamine

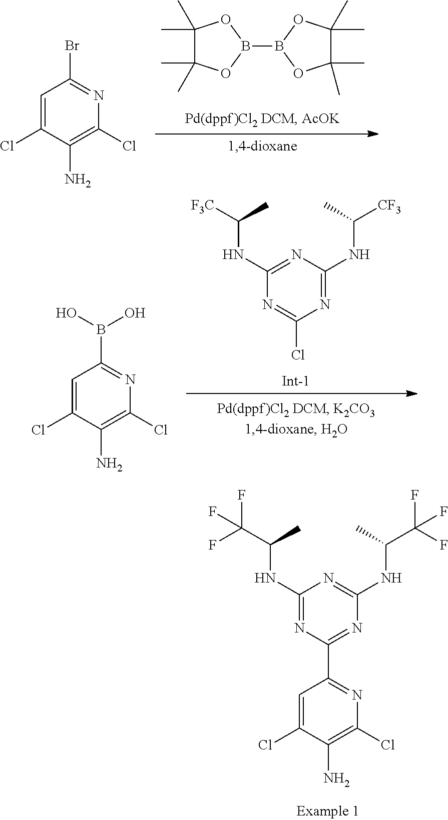

### Step 1: preparation of (5-amino-4,6-dichloropyridin-2-yl)boronic acid

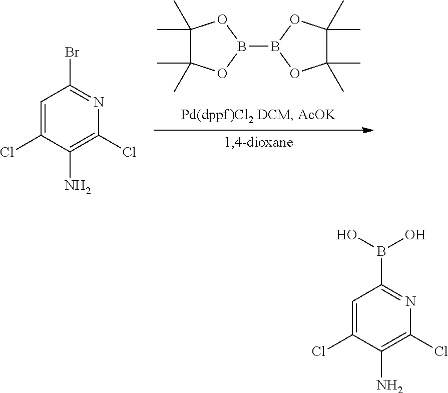

Under nitrogen protection, to compound 6-bromo-2,4-dichloropyridin-3-amine (0.50 g, 2.06 mmol) in 1,4-dioxane (5 mL) were added bis(pinacolato)diboron (0.52 g, 2.06 mmol), potassium acetate (0.41 g, 4.13 mmol) and Pd(dppf)Cl₂. DCM (0.17 g, 0.21 mmol). The reaction was stirred at 80° C. for 2 hours. After the reaction was completed, the reaction mixture was cooled to room temperature, diluted with water and extracted twice with ethyl acetate. The organic phase was concentrated under reduced pressure to obtain a target compound as a crude (0.21 g), which was used directly in the next step. LCMS [M+H⁺] 206.98.

### Step 2: preparation of (6-(5-amino-4,6-dichloropyridin-2-yl)-N²,N⁴-bis((R)-1,1,1-trifluoroprop-2-yl)-1,3,5-triazine-2,4-diamine

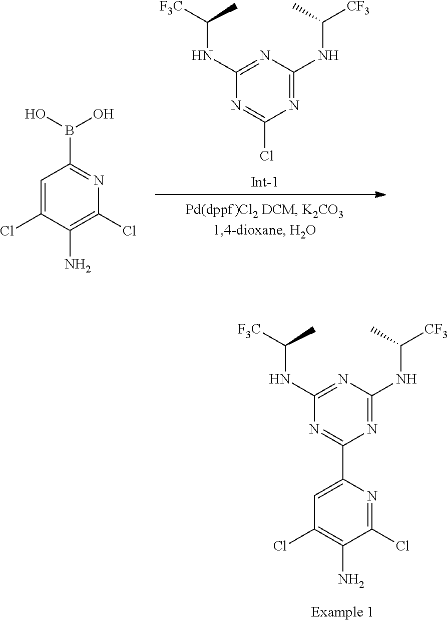 [SMILES: Nc1c(Cl)cc(B(O)O)nc1Cl ; C[C@@H](Nc1nc(Cl)nc(N[C@H](C)C(F)(F)F)n1)C(F)(F)F ; C[C@@H](Nc1nc(N[C@H](C)C(F)(F)F)nc(-c2cc(Cl)c(N)c(Cl)n2)n1)C(F)(F)F]

Under nitrogen protection, to a solution of intermediate compound Int-1 (0.3 g, 0.9 mmol) in 1,4-dioxane/water (4 mL/1 mL) were successively added (5-amino-2,6-dichloropyridin-2-yl)boronic acid (0.21 g, 1.0 mmol), K₂CO₃ (0.55 g, 4.0 mmol) and Pd(dppf)Cl₂.DCM (0.08 g, 0.1 mmol). The reaction was stirred at 90° C. for 1 hour. After LCMS detected that the reaction was completed, the reaction system was diluted with water, extracted twice with ethyl acetate, washed with a saturated aqueous sodium chloride solution, dried over anhydrous sodium sulfate and filtered. The filtrate was concentrated under reduced pressure, and the concentrated residue was subjected to Prep_TLC (n-hexane:ethyl acetate=2:1) to obtain a compound of example 1 (42 mg) as a white solid. LCMS [M+H⁺] 464.05. ¹H-NMR (500 MHz, CD₃OD) δ 8.33 (s, 1H), 5.35-5.17 (m, 1H), 5.07-4.95 (m, 1H), 1.41-1.38 (m, 6H).

### Example 2: preparation of (6-(5-amino-6-chloropyridin-2-yl)-N²,N⁴-bis((R)-1,1,1-trifluoroprop-2-yl)-1,3,5-triazine-2,4-diamine

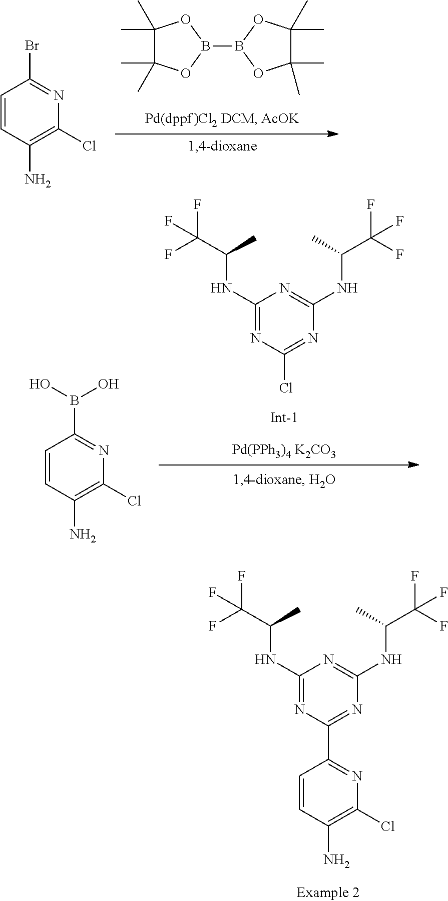

### Step 1: preparation of (5-amino-6-chloropyridin-2-yl)boronic acid

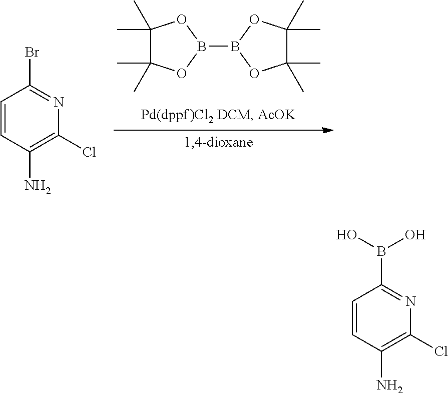

Under nitrogen protection, to a solution of compound 6-bromo-2-chloropyridin-3-amine (0.4 g, 1.93 mmol) in 1,4-dioxane were successively added bis(pinacolato)diboron (0.54 g, 2.12 mmol), potassium acetate (0.38 g, 3.86 mmol) and Pd(dppf)Cl₂ DCM (0.16 g, 0.19 mmol). The reaction was stirred at 80° C. for 3 hours. After the reaction was completed, the reaction was cooled to room temperature. The reaction system was diluted with water and extracted twice with ethyl acetate. The organic phases were combined, washed with a saturated aqueous sodium chloride solution, dried over anhydrous sodium sulfate and filtered. The filtrate was concentrated under reduced pressure. The crude was used directly in the next step. LCMS [M+H⁺] 173.02

### Step 2: preparation of (6-(5-amino-6-chloropyridin-2-yl)-N²,N⁴-bis((R)-1,1,1-trifluoroprop-2-yl)-1,3,5-triazine-2,4-diamine

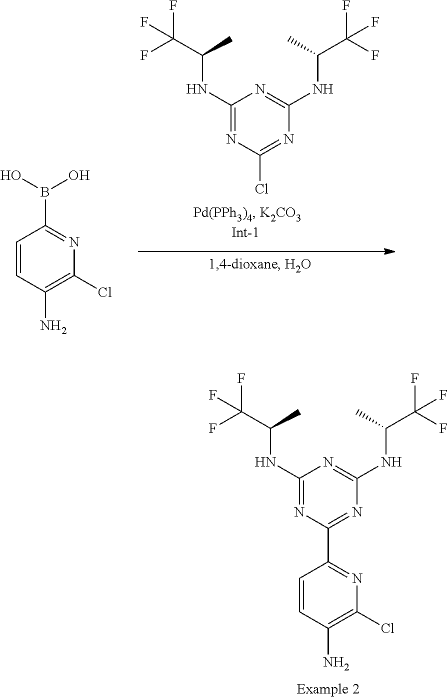 [SMILES: Nc1ccc(B(O)O)nc1Cl ; C[C@@H](Nc1nc(Cl)nc(N[C@H](C)C(F)(F)F)n1)C(F)(F)F ; C[C@@H](Nc1nc(N[C@H](C)C(F)(F)F)nc(-c2ccc(N)c(Cl)n2)n1)C(F)(F)F]

Under nitrogen protection, to a solution of intermediate compound Int-1 (0.2 g, 0.59 mmol) in 1,4-dioxane/water (3 mL/0.6 mL) were successively added (5-amino-6-chloropyridin-2-yl)boronic acid (0.10 g, 0.59 mmol), K₂CO₃ (0.16 g, 1.18 mmol) and Pd(PPh₃)₄(0.04 g, 0.03 mmol). The reaction was stirred at 85° C. for 2 hours. After LCMS detected that the reaction was completed, the reaction system was diluted with water and extracted twice with ethyl acetate. The organic phases were combined, washed with a saturated aqueous sodium chloride solution, dried over anhydrous sodium sulfate and filtered. The filtrate was concentrated under reduced pressure, and the concentrated residue was subjected to column chromatography (n-hexane:ethyl acetate=5:1) to obtain a compound of example 2 (34 mg) as a yellow solid. LCMS [M+H⁺] 430.09.

### Example 3: preparation of 6-(5-amino-6-chloro-4-methoxypyridin-2-yl)-N²,N⁴-bis((R)-1,1,1-trifluoroprop-2-yl)-1,3,5-triazine-2,4-diamine

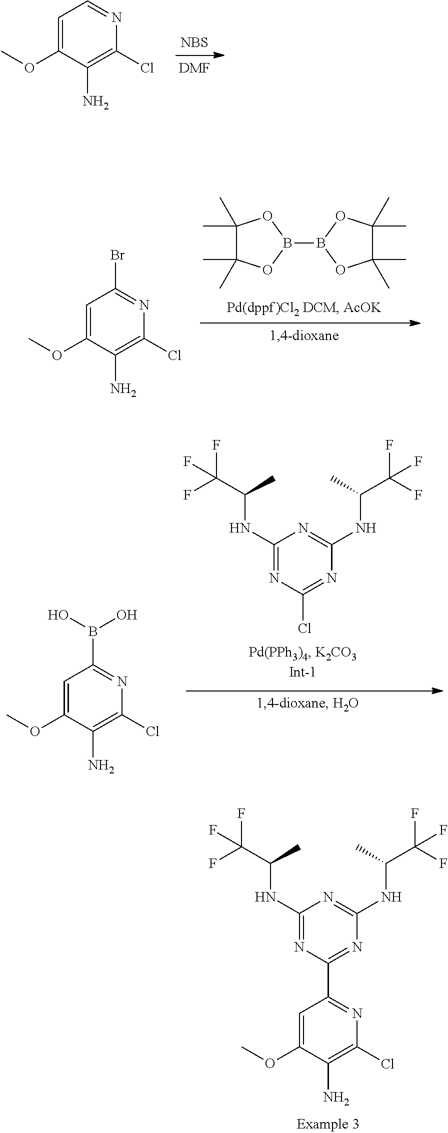

### Step 1: preparation of 6-bromo-2-chloro-4-methoxypyridin-3-amine

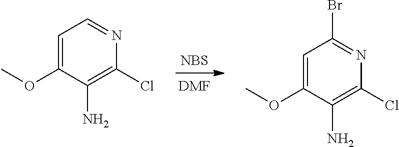

Compound 2-chloro-4-methoxypyridin-3-amine (0.5 g, 3.15 mmol) was dissolved in DMF (4 mL) and stirred at room temperature for 1 hour. After the reaction was completed, water was added to the reaction solution, and the mixture was extracted three times with ethyl acetate. The organic phases were combined. The organic phases were washed with water, dried over anhydrous sodium sulfate and filtered. The filtrate was concentrated under reduced pressure to obtain a target compound (0.6 g) as a white solid. LCMS [M+H⁺] 236.94.

### Step 2: preparation of (5-amino-6-chloro-4-methoxypyridin-2-yl)boronic acid

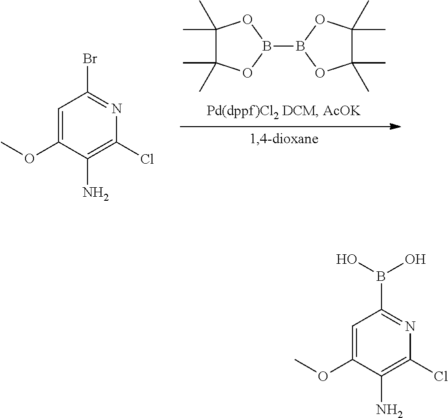

Under nitrogen protection, to a solution of compound 6-bromo-2-chloro-4-methoxypyridin-3-amine (0.1 g, 0.42 mmol) in 1,4-dioxane (3 mL) were successively added bis(pinacolato)diboron (0.11 g, 0.42 mmol), potassium acetate (0.08 g, 0.84 mmol) and Pd(dppf)Cl₂DCM (0.03 g, 0.04 mmol). The reaction was stirred at 80° C. for 2 hours. After the reaction was completed, the reaction was cooled to room temperature, diluted with water and extracted with ethyl acetate. The organic phases were dried over anhydrous sodium sulfate and filtered. The filtrate was concentrated under reduced pressure to obtain a target compound (50 mg, a crude), which was used directly in the next step. LCMS[M+H⁺] 203.03.

### Step 3: preparation of 6-(5-amino-6-chloro-4-methoxypyridin-2-yl)-N²,N⁴-bis((R)-1,1,1-trifluoroprop-2-yl)-1,3,5-triazine-2,4-diamine

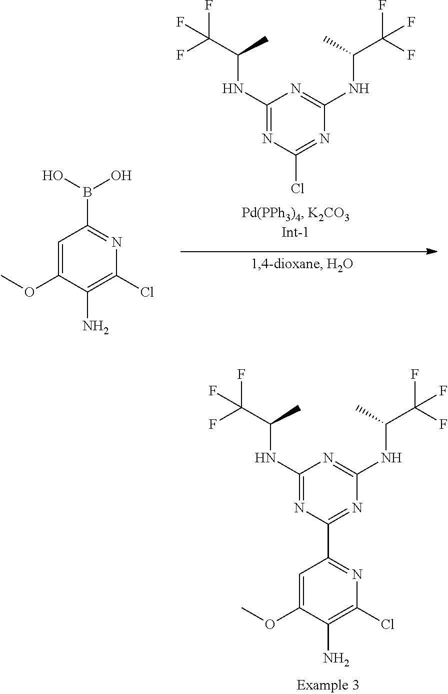 [SMILES: COc1cc(B(O)O)nc(Cl)c1N ; C[C@@H](Nc1nc(Cl)nc(N[C@H](C)C(F)(F)F)n1)C(F)(F)F ; COc1cc(-c2nc(N[C@H](C)C(F)(F)F)nc(N[C@H](C)C(F)(F)F)n2)nc(Cl)c1N]

Under nitrogen protection, to a solution of intermediate compound Int-1 (83 mg, 0.25 mmol) in 1,4-dioxane/water (4 mL/1 mL) were successively added (5-amino-6-chloro-4-methoxypyridin-2-yl)boronic acid (0.05 g, 0.25 mmol), K₂CO₃ (0.07 g, 0.49 mmol) and Pd(PPh₃)₄ (0.03 g, 0.02 mmol). The reaction was stirred at 85° C. for 2 hours. After LCMS detected that the reaction was completed, the reaction system was cooled, diluted with water and extracted twice with ethyl acetate. The organic phases were combined. The organic phases were washed with a saturated aqueous sodium chloride solution, dried over anhydrous sodium sulfate and filtered. The filtrate was concentrated under reduced pressure, and the concentrated residue was purified by Prep_TLC (n-hexane:ethyl acetate=2:1) to obtain a compound of example 3 (2 mg) as a white solid. LCMS [M+H⁺] 460.10.

### Example 4: preparation of 6-(5-amino-6-chloro-3-fluoropyridin-2-yl)-N²,N⁴-bis((R)-1,1,1-trifluoroprop-2-yl)-1,3,5-triazine-2,4-diamine

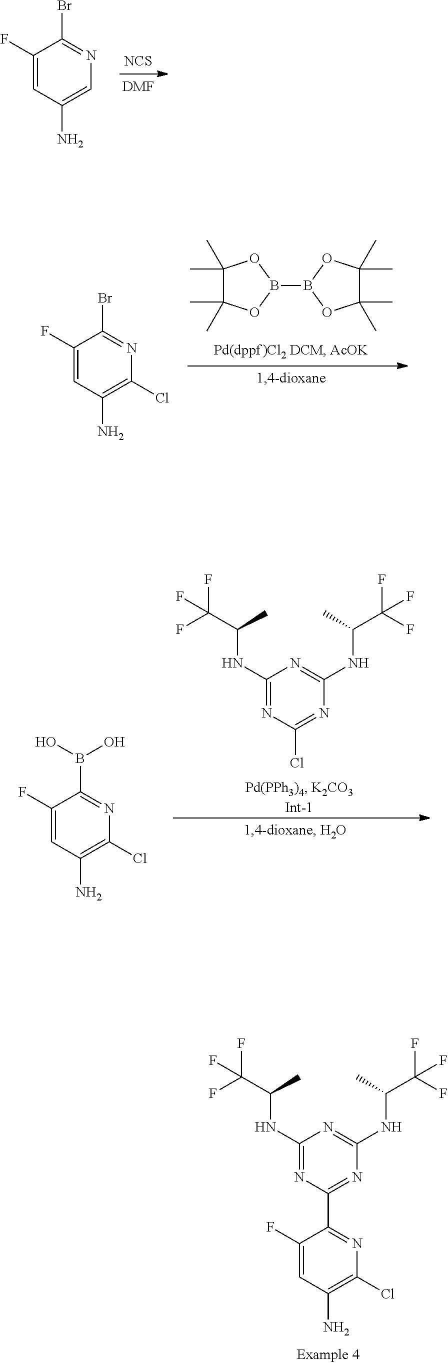

### Step 1: preparation of 6-bromo-2-chloro-5-fluoropyridin-3-amine

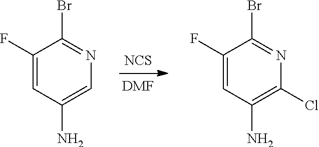 [SMILES: Nc1cnc(Br)c(F)c1 ; Nc1cc(F)c(Br)nc1Cl]

Compound 6-bromo-5-fluoropyridin-3-amine (1.4 g, 7.33 mmol) was dissolved in DMF (10 mL), NCS (0.98 g, 7.33 mmol) was added at room temperature, and the mixture was reacted at 60° C. for 1 h. After the reaction was completed, the reaction was cooled to room temperature and quenched by adding water. The reaction mixture was extracted with ethyl acetate, and the organic phase was washed by adding water, dried over anhydrous sodium sulfate and filtered. The filtrate was concentrated under reduced pressure, and the resulting crude was purified by column chromatography (ethyl acetate:n-hexane=0%-30%) to obtain a target compound (1.4 g) as a brown solid. LCMS [M+H⁺] 224.92.

### Step 2: preparation of (5-amino-6-chloro-3-fluoropyridin-2-yl)boronic acid

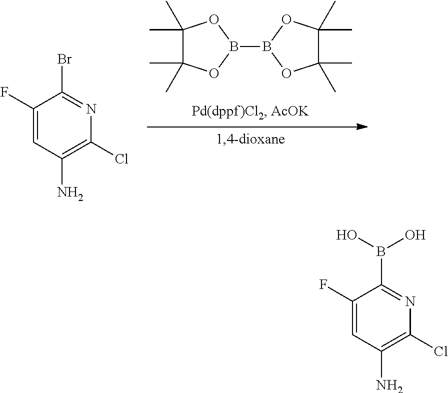

Under nitrogen protection, to a solution of compound 6-bromo-2-chloro-5-fluoropyridin-3-amine (1.4 g, 6.21 mmol) in 1,4-dioxane (30 mL) were successively added bis(pinacolato)diboron (1.58 g, 6.21 mmol), potassium acetate (1.22 g, 12.42 mmol) and Pd(dppf)Cl₂.DCM (0.51 g, 0.62 mmol). The reaction was stirred at 80° C. for 2 hours. After the reaction was completed, the reaction was cooled to room temperature, diluted with water and extracted with ethyl acetate. The organic phases were dried over anhydrous sodium sulfate and filtered. The filtrate was concentrated under reduced pressure. The resulting crude was purified by column chromatography to obtain (5-amino-6-chloro-3-fluoropyridin-2-yl)boronic acid (0.5 g) as a brown solid. LCMS [M+H⁺] 191.01.

### Step 3: preparation of 6-(5-amino-6-chloro-3-fluoropyridin-2-yl)-N²,N⁴-bis((R)-1,1,1-trifluoroprop-2-yl)-1,3,5-triazine-2,4-diamine

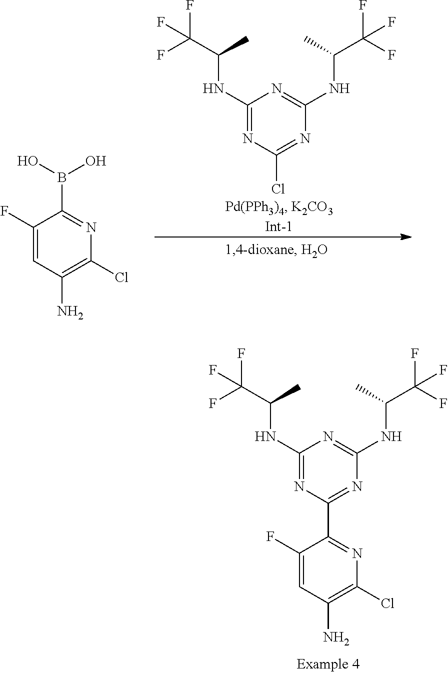 [SMILES: Nc1cc(F)c(B(O)O)nc1Cl ; C[C@@H](Nc1nc(Cl)nc(N[C@H](C)C(F)(F)F)n1)C(F)(F)F ; C[C@@H](Nc1nc(N[C@H](C)C(F)(F)F)nc(-c2nc(Cl)c(N)cc2F)n1)C(F)(F)F]

Under nitrogen protection, to a solution of intermediate compound Int-1 (0.35 g, 1.05 mmol) in 1,4-dioxane/water (4 mL/1 mL) were successively added (5-amino-6-chloro-3-fluoropyridin-2-yl)boronic acid (0.2 g, 1.05 mmol), K₂CO₃ (0.29 g, 2.1 mmol) and Pd(PPh₃)₄ (0.12 g, 0.11 mmol). The reaction was stirred at 85° C. for 2 hours. After LCMS detected that the reaction was completed, the reaction system was diluted with water, extracted twice with ethyl acetate, washed with a saturated aqueous sodium chloride solution, dried over anhydrous sodium sulfate and filtered. The filtrate was concentrated under reduced pressure, and the concentrated residue was purified by Prep_TLC (n-hexane:ethyl acetate=3:1) to obtain a compound of example 4 (40 mg) as an off-white solid. LCMS [M+H⁺] 448.08.

### Example 5: preparation of 6-(5-amino-6-chloro-4-fluoropyridin-2-yl)-N²,N⁴-bis((R)-1,1,1-trifluoropropyl-2-yl)-1,3,5-triazine-2,4-diamine

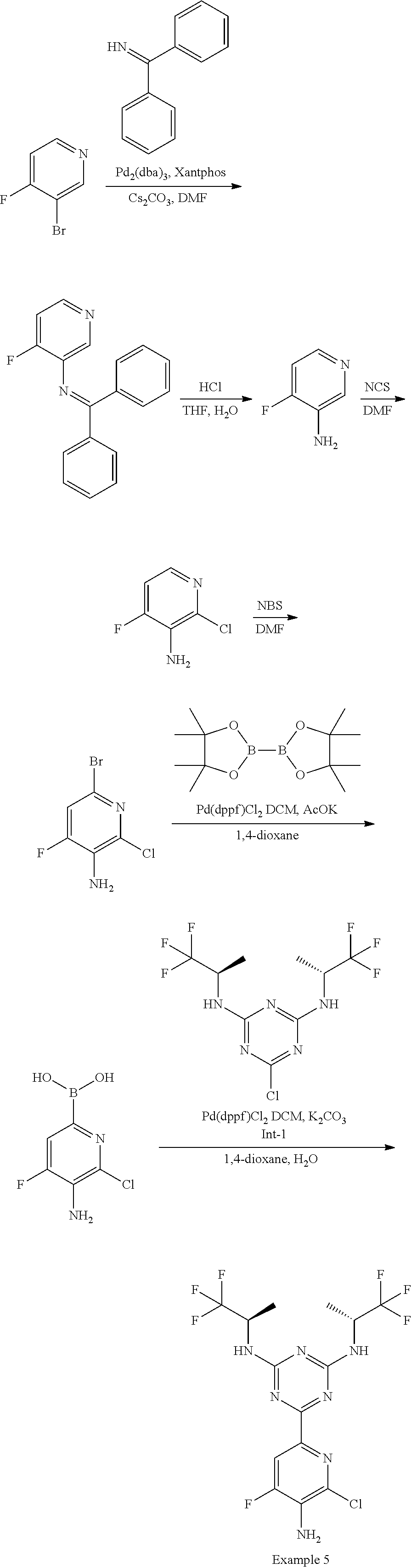

### Step 1: Synthesis of N-(4-fluoropyridin-3-yl)-1,1-diphenylmethanamine

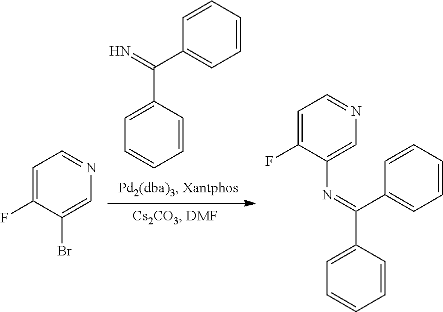

Under nitrogen protection, to compound 3-bromo-4-fluoropyridine (5.50 g, 31.25 mmol) in DMF (50 mL) were successively added diphenylmethanamine (6.80 g, 37.50 mmol), Pd₂(dba)₃ (2.86 g, 3.13 mmol), Xantphos (3.62 g, 6.25 mmol) and Cs₂CO₃ (30.55 g, 93.76 mmol). The reaction was stirred at 90° C. for 3 h. After LCMS detected that the reaction was completed, the reaction was cooled to room temperature. The reaction solution was diluted by adding ethyl acetate, washed three times with a saturated aqueous sodium chloride solution, dried over anhydrous sodium sulfate and filtered. The filtrate was concentrated under reduced pressure, and the resulting crude was purified by column chromatography (ethyl acetate:n-hexane=0%-20%) to obtain a target compound (7.50 g) as a brown oil. LCMS [M+H⁺] 277.11.

### Step 2: Synthesis of 3-amino-4-fluoropyridine

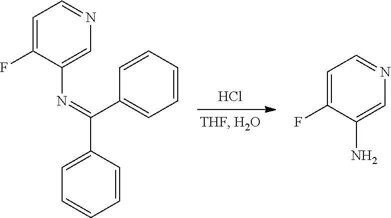 [SMILES: Fc1ccncc1N=C(c1ccccc1)c1ccccc1 ; Nc1cnccc1F]

Compound N-(4-fluoropyridin-3-yl)-1,1-diphenylmethanamine (7.50 g, 27.14 mmol) was dissolved in THF/H₂O (50 mL/50 mL), and concentrated hydrochloric acid (4.52 mL, 54.29 mmol) was added dropwise under ice bath cooling and the mixture was reacted at room temperature for 2 h. After LCMS detected that the reaction was completed, the reaction was adjusted to pH 8 with an aqueous sodium bicarbonate solution and extracted twice with ethyl acetate. The organic phases were washed with water, dried over anhydrous sodium sulfate and filtered. The filtrate was concentrated under reduced pressure, and the resulting crude was purified by column chromatography (ethyl acetate:n-hexane=0%-30%) to obtain a target compound (1.50 g) as a yellow solid. LCMS [M+H+] 113.04.

### Step 3: Synthesis of 2-chloro-3-amino-4-fluoropyridine

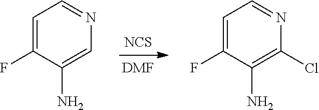 [SMILES: Nc1cnccc1F ; Nc1c(F)ccnc1Cl]

Compound 3-amino-4-fluoropyridine (1.4 g, 12.49 mmol) was dissolved in DMF (20 mL), NCS (1.67 g, 12.49 mmol) was added, and the mixture was reacted at 80° C. for 3 h. After LCMS detected that the reaction was completed, the reaction was cooled to room temperature and quenched by adding water. The reaction mixture was extracted twice with ethyl acetate, and the organic phase was washed with a saturated aqueous sodium chloride solution, dried over anhydrous sodium sulfate and filtered. The filtrate was concentrated under reduced pressure, and the resulting crude was purified by column chromatography (ethyl acetate:n-hexane=0%-30%) to obtain a target compound (0.60 g) as a brown solid. LCMS [M+H⁺] 147.00.

### Step 4: Synthesis of 6-bromo-2-chloro-4-fluoropyridin-3-amine

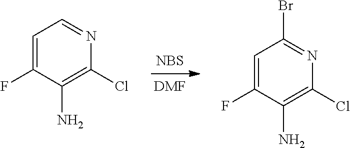 [SMILES: Nc1c(F)ccnc1Cl ; Nc1c(F)cc(Br)nc1Cl]

Compound 6-bromo-2-chloro-4-fluoropyridin-3-amine (600 mg, 4.09 mmol) was dissolved in DMF (10 mL), NBS (802 mg, 4.5 mmol) was added at room temperature, and the reaction was continued for 3 h. The reaction was quenched by adding water. The reaction mixture was extracted twice with ethyl acetate, and the organic phase was washed with a saturated aqueous sodium chloride solution, dried over anhydrous sodium sulfate and filtered. The filtrate was concentrated under reduced pressure, and the resulting crude was purified by column chromatography to obtain a target compound (767 mg) as a yellow solid. LCMS [M+H⁺] 224.92.

### Step 5: Synthesis of (5-amino-6-chloro-4-fluoropyridin-2-yl)boronic acid

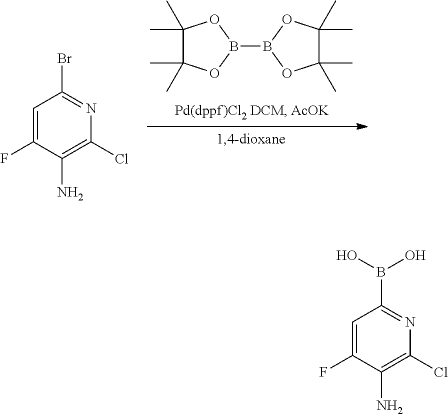

Under nitrogen protection, to a solution of compound 6-bromo-2-chloro-4-fluoropyridin-3-amine (767 mg, 3.4 mmol) in 1,4-dioxane (10 mL) were successively added bis(pinacolato)diboron (950 mg, 3.74 mmol), potassium acetate (1.0 g, 10.21 mmol) and Pd(dppf)Cl₂ DCM (277.82 mg, 0.34 mmol). The reaction was stirred at 80° C. for 3 h. After LCMS detected that the reaction was completed, the reaction mixture was concentrated under reduced pressure. The resulting crude was purified by column chromatography (ethyl acetate:n-hexane=0%-40%) to obtain a target compound (100 mg) as a light-yellow solid. LCMS [M+H+]191.01.

### Step 6: Synthesis of 6-(5-amino-6-chloro-4-fluoropyridin-2-yl)-N²,N⁴-bis((R)-1,1,1-trifluoroprop-2-yl)-1,3,5-triazine-2,4-diamine

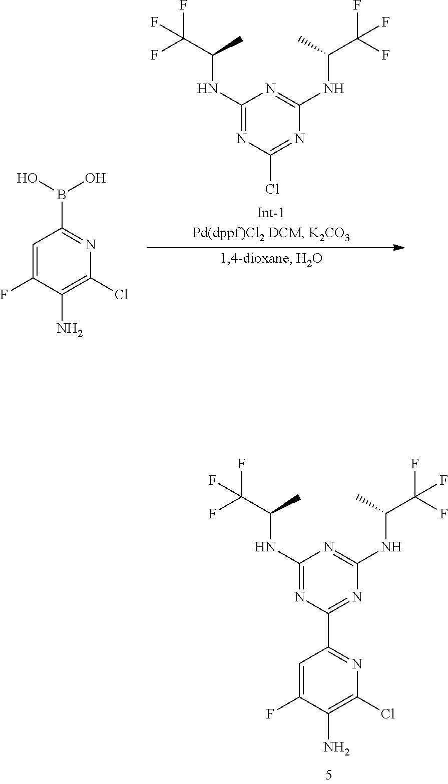 [SMILES: Nc1c(F)cc(B(O)O)nc1Cl ; C[C@@H](Nc1nc(Cl)nc(N[C@H](C)C(F)(F)F)n1)C(F)(F)F ; C[C@@H](Nc1nc(N[C@H](C)C(F)(F)F)nc(-c2cc(F)c(N)c(Cl)n2)n1)C(F)(F)F]

Under nitrogen protection, to a solution of compound (5-amino-6-chloro-4-fluoropyridin-2-yl)boronic acid (100 mg, 0.53 mmol) in 1,4-dioxane/water (10.00 mL/2.00 mL) were successively added compound Int-1 (177 mg, 0.53 mmol), [PdCl₂(dppf)]CH₂Cl₂ (42.87 mg, 0.05 mmol) and K₂CO₃ (218 mg, 1.58 mmol). The reaction was stirred at 80° C. for 3 h. After LCMS detected that the reaction was completed, the reaction compound was concentrated under reduced pressure, and the resulting crude was purified by Prep-HPLC (Column: Luna-C18(3)-10-100; Size: 30*250 nm, Conditions: acetonitrile/water (0.1% formic acid) 35%-65%, 20 min, UV: 308 nm), to obtain a compound of example 5 (4.5 mg) as a white solid. LCMS [M+H⁺] 448.08. ¹H-NMR (500 MHz, CD₃OD) δ 8.13-8.10 (d, J=15 MHz, 1H), 5.33-5.24 (m, 1H), 5.02-4.94 (m, 1H), 1.41-1.37 (m, 6H).

### Example 6: preparation of 6-(5-amino-4-bromo-6-chloropyridin-2-yl)-N²,N⁴-bis((R)-1,1,1-trifluoropropyl-2-yl)-1,3,5-triazine-2,4-diamine

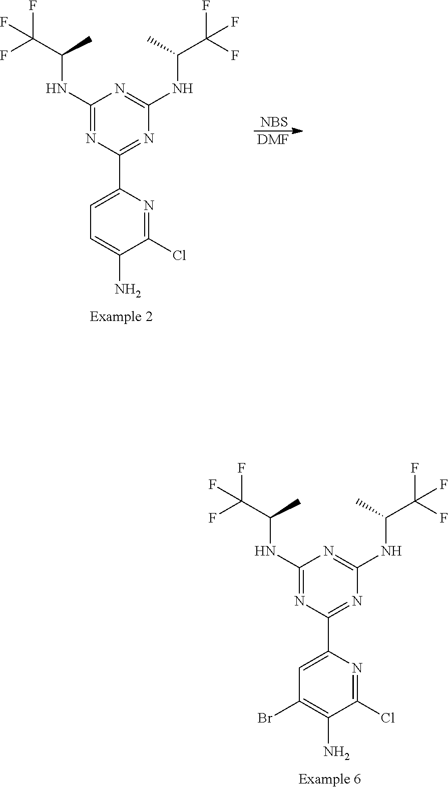

The compound of example 2 (1.20 g, 2.79 mmol) was dissolved in DMF (10 mL), NBS (0.55 g, 3.07 mmol) was added at room temperature, and the mixture was reacted at room temperature for 2 h. After LCMS detected that the reaction was completed, the reaction was quenched by adding water. The reaction mixture was extracted twice with ethyl acetate, and the organic phase was washed with water, dried over anhydrous sodium sulfate and filtered. The filtrate was concentrated under reduced pressure, and the resulting crude was purified by column chromatography (ethyl acetate:n-hexane=0%-60%) to obtain a target compound (0.40 g) as a brown solid. LCMS [M+H⁺] 508.00.

### Example 7: preparation of 6-(5-amino-6-chloro-4-methylpyridin-2-yl)-N²,N⁴-bis((R)-1,1,1-trifluoropropyl-2-yl)-1,3,5-triazine-2,4-diamine

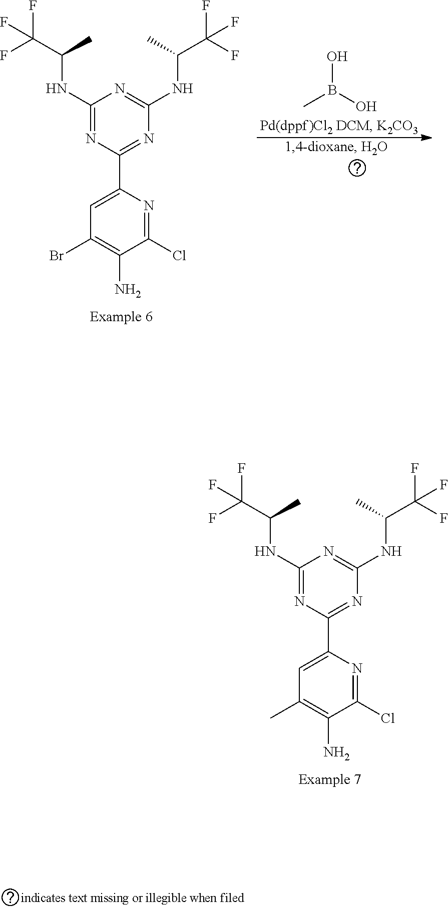

Under nitrogen protection, to a solution of compound 6-(5-amino-4-bromo-6-chloropyridin-2-yl)-N²,N⁴-bis((R)-1,1,1-trifluoroprop-2-yl)-1,3,5-triazine-2,4-diamine (150 mg, 0.29 mmol) in 1,4-dioxane/water (1 mL/0.2 mL) were successively added methylboronic acid (38 mg, 0.29 mmol), [PdCl₂(dppf)]CH₂Cl₂ (24 mg, 0.03 mmol) and potassium carbonate (122 mg, 0.88 mmol). The reaction was stirred at 80° C. for 16 h. After LCMS detected that the reaction was completed, the reaction was cooled to room temperature. The reaction mixture was diluted by adding ethyl acetate, and washed with a saturated aqueous sodium chloride solution. The organic phase was dried over anhydrous sodium sulfate and filtered. The filtrate was concentrated under reduced pressure, and the resulting crude was purified by Prep-TLC (ethyl acetate: n-hexane=1:3) to obtain a compound of example 7 (33.4 mg) as a white solid. LCMS [M+H⁺] 444.11.

### Example 8: preparation of 6-(5-amino-6-chloro-4-(difluoromethyl)pyridin-2-yl)-N²,N⁴-bis((R)-1,1,1-trifluoroprop-2-yl)-1,3,5-triazine-2,4-diamine

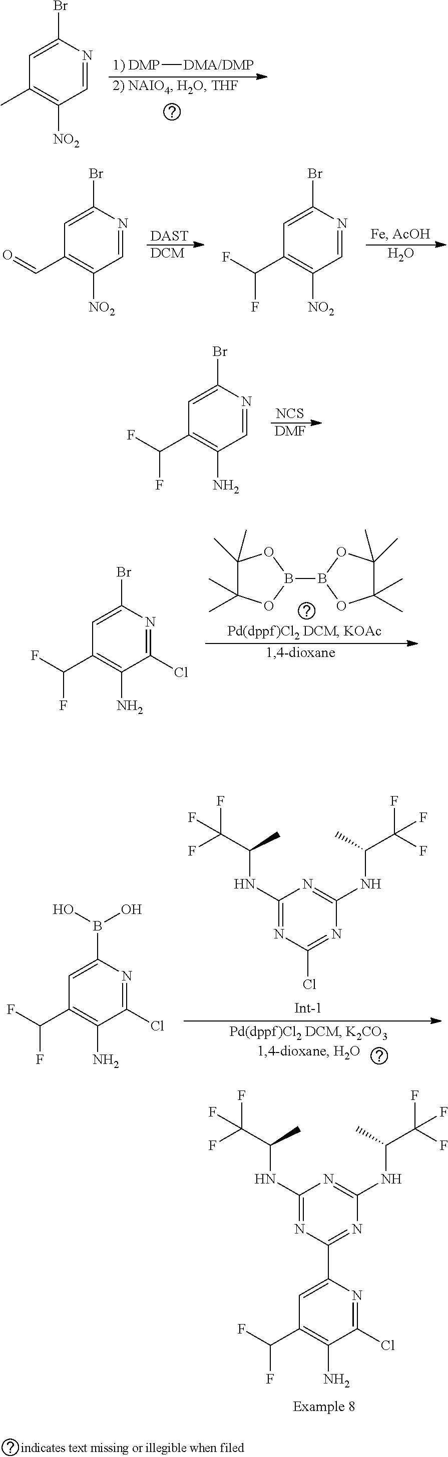

### Step 1: Synthesis of 2-bromo-5-nitroisonicotinaldehyde

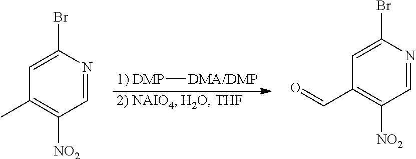

Compound 2-bromo-4-methyl-5-nitropyridine (5.0 g, 23.04 mmol) was dissolved in 30 mL of DMF, and DMF-DMA (5.5 g, 46.08 mmol) was added dropwise under ice bath. After the dropwise addition was completed, the reaction solution was stirred at 90° C. for 2 hours. After the reaction was completed, the reaction was cooled to room temperature and dissolved in 50 mL of THF. Then, an aqueous solution (50 mL) of NaIO₄ (11.9 g, 55.13 mmol) was added to the above-mentioned reaction solution under ice bath, and the mixture was stirred at room temperature for another 3 hours. After the reaction was completed, the reaction mixture was extracted three times with ethyl acetate. The organic phases were combined, washed with saturated brine, dried over anhydrous sodium sulfate and filtered by suction. The filtrate was concentrated under reduced pressure, and the crude was subjected to column chromatography (n-hexane:ethyl acetate=3:1) to obtain a target compound (3.4 g) as a brown oil. LCMS [M+H⁺]230.93

### Step 2: Synthesis of 2-bromo-4-(difluoromethyl)-5-nitropyridine

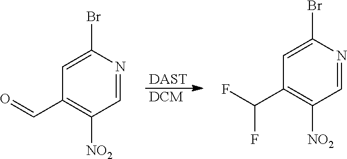

Compound 2-bromo-5-nitroisonicotinaldehyde (3.4 g, 14.72 mmol) was dissolved in 30 mL of dichloromethane, and DAST (7.12 g, 44.16 mmol) was added dropwise under ice bath. After the dropwise addition was completed, the mixture was stirred at room temperature for 3 hours. After the reaction was completed, the reaction solution was adjusted to a neutral pH with saturated sodium bicarbonate and extracted three times with dichloromethane. The organic phases were combined, washed with saturated brine, dried over anhydrous sodium sulfate and filtered by suction. The filtrate was concentrated under reduced pressure to obtain a target compound (3.0 g).

LCMS [M+H⁺] 252.93

### Step 3: Synthesis of 6-bromo-4-(difluoromethyl)pyridin-3-amine

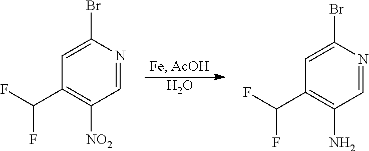

Compound 2-bromo-4-(difluoromethyl)-5-nitropyridine (3.0 g, 11.86 mmol) was dissolved in acetic acid/water (10 mL/2 mL), an iron powder (1.99 g, 35.57 mmol) was added to the above-mentioned solution, and the mixture was stirred at room temperature for 2 hours. After the reaction was completed, the reaction mixture was filtered by suction. The filtrate was concentrated under reduced pressure, and the crude was subjected to column chromatography (n-hexane:ethyl acetate=3:1) to obtain a target compound (1.7 g) as a brown solid. LCMS [M+H⁺] 222.96

### Step 4: Synthesis of 6-bromo-2-chloro-4-(difluoromethyl)pyridin-3-amine

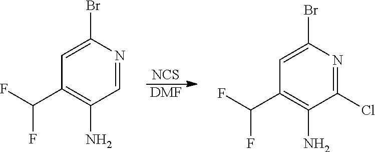

Compound 6-bromo-4-(difluoromethyl)pyridin-3-amine (1.7 g, 7.62 mmol) was dissolved in DMF (10 mL), NCS (1.02 g, 7.62 mmol) was added at room temperature, and the reaction was continued for 1 h. The reaction was quenched by adding water. The reaction mixture was extracted twice with ethyl acetate, and the organic phase was washed with a saturated aqueous sodium chloride solution, dried over anhydrous sodium sulfate and filtered. The filtrate was concentrated under reduced pressure to obtain a target compound (1.5 g) as a brown oil. LCMS [M+H+] 256.92.

### Step 5: Synthesis of (5-amino-6-chloro-4-(difluoromethyl)pyridin-2-yl)boronic acid

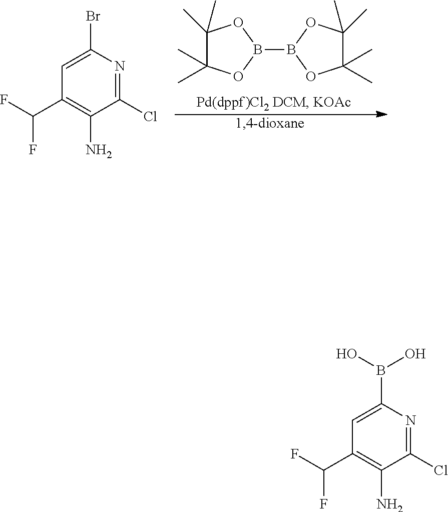

Under nitrogen protection, to a solution of compound 6-bromo-2-chloro-4-(difluoromethyl)pyridin-3-amine (1.0 g, 3.88 mmol) in 1,4-dioxane (20 mL) were successively added bis(pinacolato)diboron (990 mg, 3.88 mmol), potassium acetate (760 g, 7.77 mmol) and Pd(dppf)Cl₂ DCM (320 mg, 0.39 mmol). The reaction was stirred at 85° C. for 4 h. After LCMS detected that the reaction was completed, the reaction mixture was concentrated under reduced pressure, and the resulting crude was used directly in the next step. LCMS [M+H⁺]223.02

### Step 6: preparation of 6-(5-amino-6-chloro-4-(difluoromethyl)pyridin-2-yl)-N²,N⁴-bis((R)-1,1,1-trifluoroprop-2-yl)-1,3,5-triazine-2,4-diamine

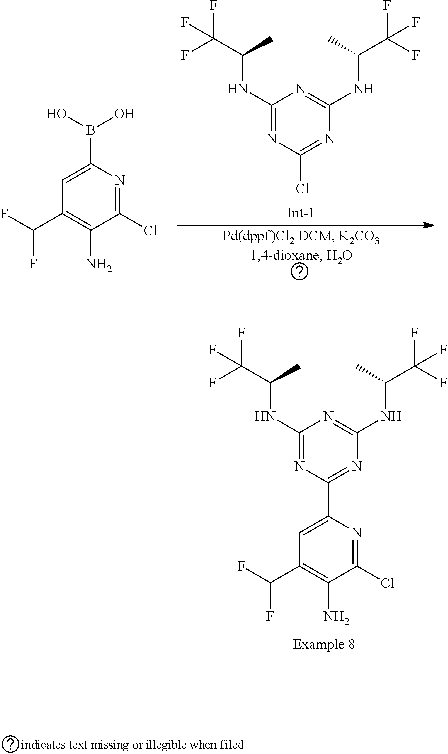 [SMILES: Nc1c(C(F)F)cc(B(O)O)nc1Cl ; C[C@@H](Nc1nc(Cl)nc(N[C@H](C)C(F)(F)F)n1)C(F)(F)F ; C[C@@H](Nc1nc(N[C@H](C)C(F)(F)F)nc(-c2cc(C(F)F)c(N)c(Cl)n2)n1)C(F)(F)F]

Under nitrogen protection, to a solution of compound (5-amino-6-chloro-4-(difluoromethyl)pyridin-2-yl)boronic acid (850 mg, 3.82 mmol) in 1,4-dioxane/H₂O (15.00 mL/3.00 mL) were successively added compound Int-1 (1.16 g, 3.44 mmol), [PdCl₂(dppf)]CH₂Cl₂ (310 mg, 0.38 mmol) and K₂CO₃ (1.06 g, 7.64 mmol). The reaction was stirred at 85° C. for 2 h. After LCMS detected that the reaction was completed, the reaction compound was concentrated under reduced pressure, and the resulting crude was purified by Prep-TLC (n-hexane:ethyl acetate=2:1) to obtain a compound of example 8 (120 mg) as a white solid. LCMS [M+H⁺] 480.09. ¹H-NMR (500 MHz, CD₃OD) δ8.38 (s, 1H), 7.10-6.83 (m, 1H), 5.33-5.24 (m, 1H), 5.02-4.92 (m, 1H), 1.41-1.38 (m, 6H).

### Example 9: preparation of 6-(5-amino-6-chloro-4-vinylpyridin-2-yl)-N²,N⁴-bis((R)-1,1,1-trifluoropropyl-2-yl)-1,3,5-triazine-2,4-diamine

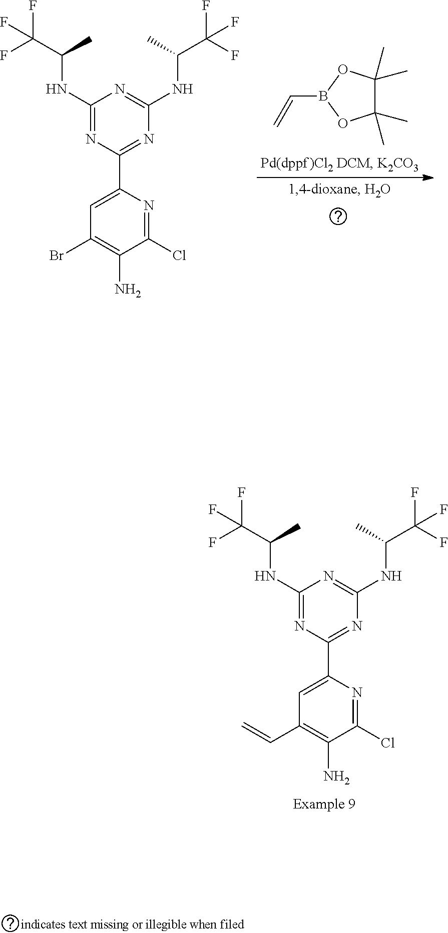 [SMILES: C[C@@H](Nc1nc(N[C@H](C)C(F)(F)F)nc(-c2cc(Br)c(N)c(Cl)n2)n1)C(F)(F)F ; C=CB1OC(C)(C)C(C)(C)O1 ; C=Cc1cc(-c2nc(N[C@H](C)C(F)(F)F)nc(N[C@H](C)C(F)(F)F)n2)nc(Cl)c1N]

Under nitrogen protection, to a solution of compound 6-(5-amino-4-bromo-6-chloropyridin-2-yl)-N²,N⁴-bis((R)-1,1,1-trifluoroprop-2-yl)-1,3,5-triazine-2,4-diamine (150 mg, 0.29 mmol) in 1,4-dioxane/water (10 mL/1 mL) were successively added vinylboronic acid pinacol ester (68 mg, 0.44 mmol),[PdCl₂(dppf)]CH₂Cl₂ (24 mg, 0.03 mmol) and potassium carbonate (122 mg, 0.88 mmol). The reaction was stirred at 80° C. for 3 h. After the reaction was completed, the reaction mixture was concentrated under reduced pressure, and the resulting crude was purified by column chromatography (ethyl acetate:n-hexane=0%-25%) to obtain a compound of example 9 (16.7 mg) as a white solid. LCMS [M+H⁺] 456.11.

### Example 10: preparation of 6-(4,6-bis(((R)-1,1,1-trifluoroprop-2-yl)amine)-1,3,5-triazin-2-yl)-2-chloro-4-fluoropyridin-3-ol

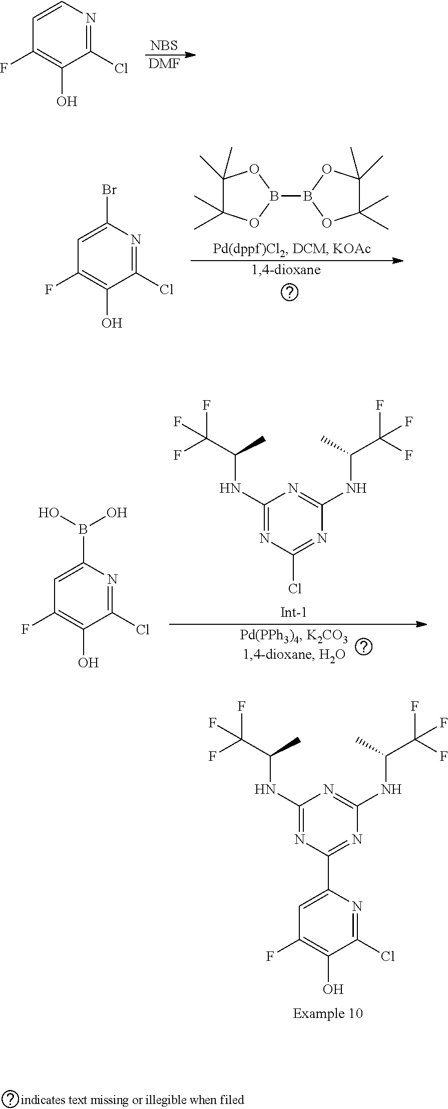

### Step 1: Synthesis of 6-bromo-chloro-4-fluoropyridin 3 ol

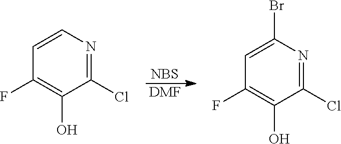

Compound 2-chloro-4-fluoropyridin-3-ol (300 mg, 2.03 mmol) was dissolved in DMF (3 mL), NBS (360 mg, 2.03 mmol) was added at room temperature, and the reaction was continued for 1 h. The reaction was quenched by adding water. The reaction mixture was extracted twice with ethyl acetate, and the organic phase was washed with a saturated aqueous sodium chloride solution, dried over anhydrous sodium sulfate and filtered. The filtrate was concentrated under reduced pressure to obtain a target compound (380 mg) as a yellow solid. LCMS [M+H+] 225.90.

### Step 2: Synthesis of (6-chloro-4-fluoro-5-hydroxypyridin-2-yl)boronic acid

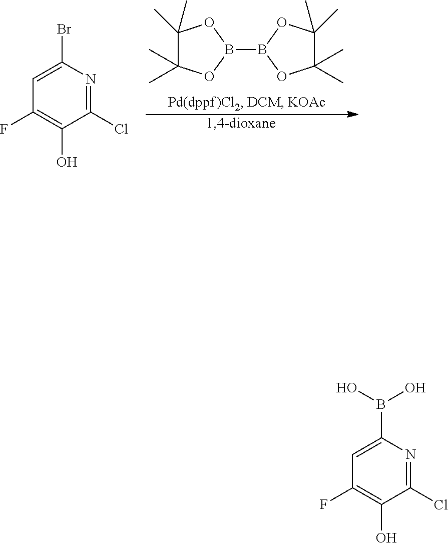

Under nitrogen protection, to a solution of compound 6-bromo-2-chloro-4-fluoropyridin-3-ol (380 mg, 1.68 mmol) in 1,4-dioxane (5 mL) were successively added bis(pinacolato)diboron (430 mg, 1.68 mmol), potassium acetate (330 g, 3.36 mmol) and Pd(dppf)Cl₂ DCM (120 mg, 0.17 mmol). The reaction was stirred at 80° C. for 2 h. After LCMS detected that the reaction was completed, the reaction mixture was concentrated under reduced pressure, and the resulting crude was used directly in the next step. LCMS [M+H+] 192.00

### Step 3: preparation of 6-(4,6-bis(((R)-1,1,1-trifluoroprop-2-yl)amine)-1,3,5-triazin-2-yl)-2-chloro-4-fluoropyridin-3-ol

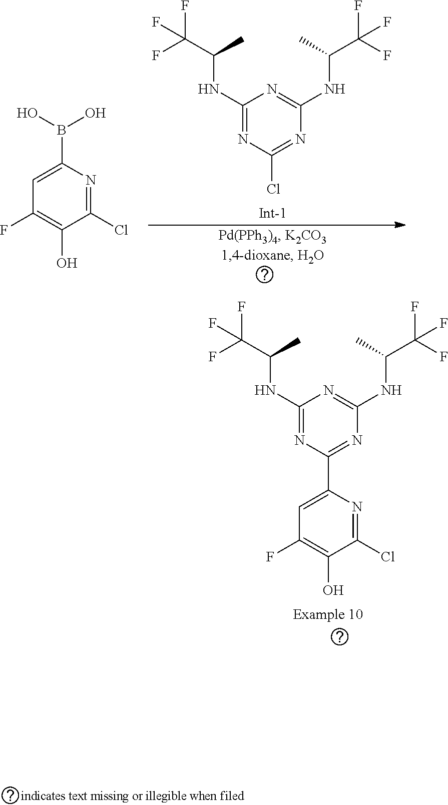 [SMILES: OB(O)c1cc(F)c(O)c(Cl)n1 ; C[C@@H](Nc1nc(Cl)nc(N[C@H](C)C(F)(F)F)n1)C(F)(F)F ; C[C@@H](Nc1nc(N[C@H](C)C(F)(F)F)nc(-c2cc(F)c(O)c(Cl)n2)n1)C(F)(F)F]

Under nitrogen protection, to a solution of compound (5-amino-6-chloro-4-fluoropyridin-2-yl)boronic acid (226 mg, 1.18 mmol) in 1,4-dioxane/H₂O (4.00 mL/1.00 mL) were successively added compound Int-1 (400 mg, 1.18 mmol), Pd(PPh₃)₄(68 mg, 0.05 mmol) and K₂CO₃ (327 mg, 2.37 mmol). The reaction was stirred at 80° C. for 3 h. After LCMS detected that the reaction was completed, the reaction compound was concentrated under reduced pressure, and the resulting crude was purified by Prep-TLC (n-hexane:ethyl acetate=2:1) to obtain a compound of example 10 (7.4 mg) as a brown solid. LCMS [M+H⁺] 449.06.

With reference to the methods in examples 1, 3-6, 9 and 10, the example compounds in the table were synthesized by selecting the corresponding intermediates and reagents.

| Example | Structural formula | Name | [M + H⁺] |
|---|---|---|---|
| 11 | 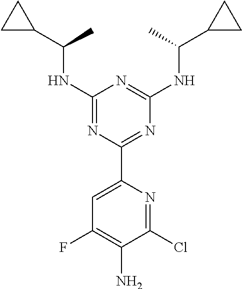 [SMILES: C[C@@H](Nc1nc(N[C@H](C)C2CC2)nc(-c2cc(F)c(N)c(Cl)n2)n1)C1CC1] | 6-(5-amino-6-chloro-4- fluoropyridin-2-yl)-N²,N⁴- bis((R)-1-cyclopropylethyl)-1,3,5- triazine-2,4-diamine | 392.17 |
| 12 | 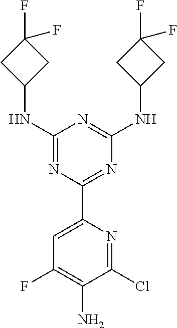 [SMILES: Nc1c(F)cc(-c2nc(NC3CC(F)(F)C3)nc(NC3CC(F)(F)C3)n2)nc1Cl] | 6-(5-amino-6-chloro-4- fluoropyridin-2-yl)-N²,N⁴-bis(3,3- difluorocyclobutyl)-1,3,5-triazine- 2,4-diamine | 436.10 |
| 13 | 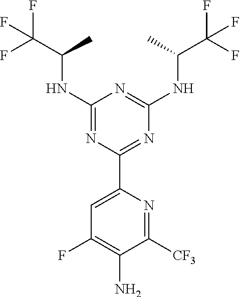 [SMILES: C[C@@H](Nc1nc(N[C@H](C)C(F)(F)F)nc(-c2cc(F)c(N)c(C(F)(F)F)n2)n1)C(F)(F)F] | 6-(5-amino-4-fluoro-6- (trifluoromethyl)pyridin-2-yl)- N²,N⁴-bis((R)-1,1,1-trifluoroprop- 2-yl)-1,3,5-triazine-2,4-diamine | 482.11 |
| 14 | 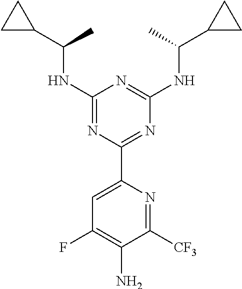 [SMILES: C[C@@H](Nc1nc(N[C@H](C)C2CC2)nc(-c2cc(F)c(N)c(C(F)(F)F)n2)n1)C1CC1] | 6-(5-amino-4-fluoro-6- (trifluoromethyl)pyridin-2-yl)- N²,N⁴-bis((R)-1- cyclopropylethyl)-1,3,5-triazine- 2,4-diamine | 426.20 |
| 15 | 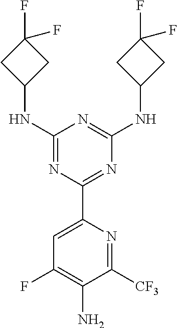 [SMILES: Nc1c(F)cc(-c2nc(NC3CC(F)(F)C3)nc(NC3CC(F)(F)C3)n2)nc1C(F)(F)F] | 6-(5-amino-4-fluoro-6- (trifluoromethyl)pyridin-2-yl)- N²,N⁴-bis(3,3- difluorocyclobutyl)-1,3,5-triazine- 2,4-diamine | 470.13 |
| 16 | 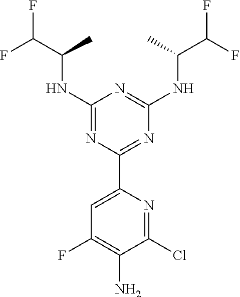 [SMILES: C[C@@H](Nc1nc(N[C@H](C)C(F)F)nc(-c2cc(F)c(N)c(Cl)n2)n1)C(F)F] | 6-(5-amino-6-chloro-4- fluoropyridin-2-yl)-N²,N⁴- bis((R)-1,1-difluoroprop-2-yl)- 1,3,5-triazine-2,4-diamine | 412.10 |
| 17 | 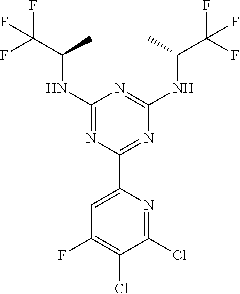 [SMILES: C[C@@H](Nc1nc(N[C@H](C)C(F)(F)F)nc(-c2cc(F)c(Cl)c(Cl)n2)n1)C(F)(F)F] | 6-(5,6-dichloro-4-fluoropyridin-2- yl)-N²,N⁴-bis((R)-1,1,1- trifluoroprop-2-yl)-1,3,5-triazine- 2,4-diamine | 467.03 |
| 18 | 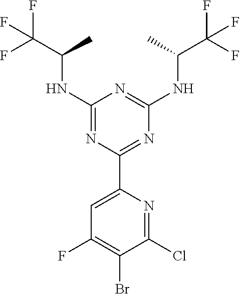 [SMILES: C[C@@H](Nc1nc(N[C@H](C)C(F)(F)F)nc(-c2cc(F)c(Br)c(Cl)n2)n1)C(F)(F)F] | 6-(5-bromo-6-chloro-4- fluoropyridin-2-yl)-N²,N⁴- bis((R)-1,1,1-trifluoroprop-2-yl)- 1,3,5-triazine-2,4-diamine | 510.98 |
| 19 | 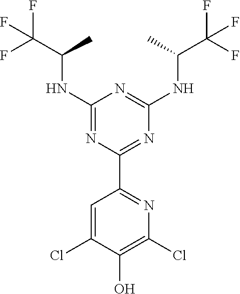 [SMILES: C[C@@H](Nc1nc(N[C@H](C)C(F)(F)F)nc(-c2cc(Cl)c(O)c(Cl)n2)n1)C(F)(F)F] | 6-(4,6-dichloro-5hydroxylpyridin- 2-yl)-N²,N⁴-bis((R)-1,1,1- trifluoroprop-2-yl)-1,3,5-triazine- 2,4-diamine | 465.04 |
| 20 | 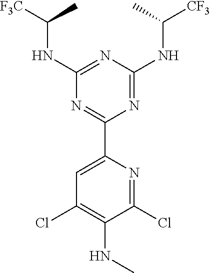 [SMILES: CNc1c(Cl)cc(-c2nc(N[C@H](C)C(F)(F)F)nc(N[C@H](C)C(F)(F)F)n2)nc1Cl] | 6-(4,6-dichloro-5- methylaminopyridin-2-yl)-N²,N⁴- bis((R)-1,1,1-trifluoroprop-2-yl)- 1,3,5-triazine-2,4-diamine | 478.07 |
| 21 | 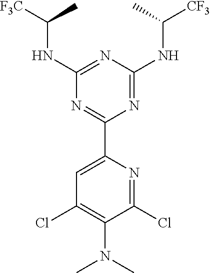 [SMILES: C[C@@H](Nc1nc(N[C@H](C)C(F)(F)F)nc(-c2cc(Cl)c(N(C)C)c(Cl)n2)n1)C(F)(F)F] | 6-(4,6-dichloro-5-dimethylamino pyridin-2-yl)-N²,N⁴-bis((R)-1,1,1- trifluoroprop-2-yl)-1,3,5-triazine- 2,4-diamine | 492.08 |
| 22 | 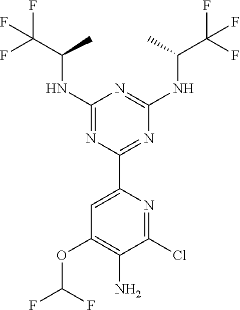 [SMILES: C[C@@H](Nc1nc(N[C@H](C)C(F)(F)F)nc(-c2cc(OC(F)F)c(N)c(Cl)n2)n1)C(F)(F)F] | 6-(5-amino-6-chloro-4- (difluoromethoxy)pyridin-2-yl)- N²,N⁴-bis((R)-1,1,1- trifluoropropyl-2-yl)-1,3,5- triazine-2,4-diamine | 496.08 |
| 23 | 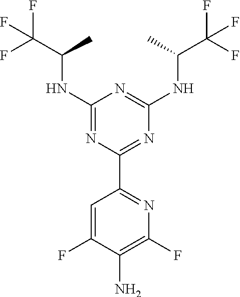 [SMILES: C[C@@H](Nc1nc(N[C@H](C)C(F)(F)F)nc(-c2cc(F)c(N)c(F)n2)n1)C(F)(F)F] | 6-(5-amino-4,6-difluoropyridin-2- yl)-N²,N⁴-bis((R)-1,1,1- trifluoroprop-2-yl)-1,3,5-triazine- 2,4-diamine | 432.11 |
| 24 | 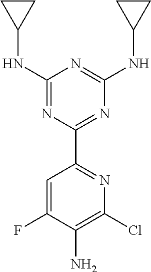 [SMILES: Nc1c(F)cc(-c2nc(NC3CC3)nc(NC3CC3)n2)nc1Cl] | 6-(6-chloro-4-fluoro-5- aminopyridin-2-yl)-N²,N⁴- dicyclopropyl-1,3,5-triazine-2,4- diamine | 336.11 |
| 25 | 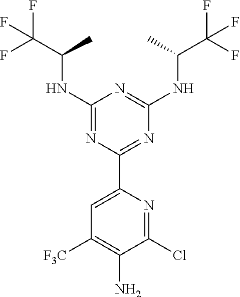 [SMILES: C[C@@H](Nc1nc(N[C@H](C)C(F)(F)F)nc(-c2cc(C(F)(F)F)c(N)c(Cl)n2)n1)C(F)(F)F] | 6-(6-chloro-5-amino-4- (trifluoromethyl)pyridin-2-yl)- N²,N⁴-bis((R)-1,1,1- trifluoropropyl-2-yl)-1,3,5- triazine-2,4-diamine | 498.08 |
| 26 | 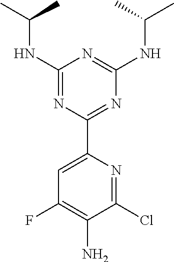 | 6-(5-amino-6-chloro-4- fluoropyridin-2-yl)-N²,N⁴- diisopropyl-1,3,5-triazine-2,4- diamine | 340.14 |
| 27 | 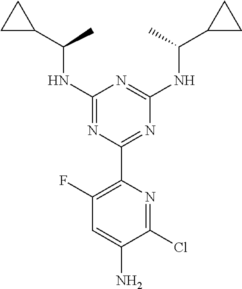 [SMILES: C[C@@H](Nc1nc(N[C@H](C)C2CC2)nc(-c2nc(Cl)c(N)cc2F)n1)C1CC1] | 6-(5-amino-6-chloro-3- fluoropyridin-2-yl)-N²,N⁴-bis(R)- 1-cyclopropylethyl)-1,3,5- triazine-2,4-diamine | 392.17 |
| 28 | 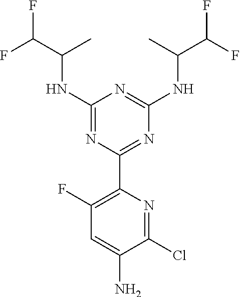 [SMILES: CC(Nc1nc(NC(C)C(F)F)nc(-c2nc(Cl)c(N)cc2F)n1)C(F)F] | 6-(5-amino-6-chloro-3- fluoropyridin-2-yl)-N²,N⁴-bis(1,1- difluoroprop-2-yl)-1,3,5-triazine- 2,4-diamine | 412.10 |
| 29 | 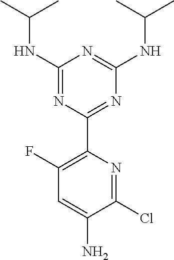 [SMILES: CC(C)Nc1nc(NC(C)C)nc(-c2nc(Cl)c(N)cc2F)n1] | 6-(5-amino-6-chloro-3- fluoropyridin-2-yl)-N²,N⁴- diisopropyl-1,3,5-triazine-2,4- diamine | 340.14 |
| 30 | 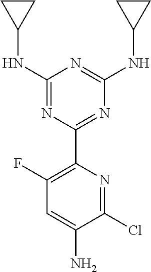 [SMILES: Nc1cc(F)c(-c2nc(NC3CC3)nc(NC3CC3)n2)nc1Cl] | 6-(5-amino-6-chloro-3- fluoropyridin-2-yl)-N²,N⁴- dicyclopropyl-1,3,5-triazine-2,4- diamine | 336.11 |
| 31 | 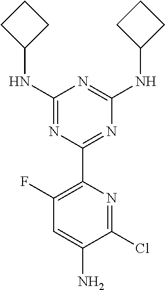 [SMILES: Nc1cc(F)c(-c2nc(NC3CCC3)nc(NC3CCC3)n2)nc1Cl] | 6-(5-amino-6-chloro-3- fluoropyridin-2-yl)-N²,N⁴- dicyclobutyl-1,3,5-triazine-2,4- diamine | 364.14 |
| 32 | 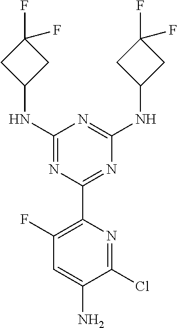 [SMILES: Nc1cc(F)c(-c2nc(NC3CC(F)(F)C3)nc(NC3CC(F)(F)C3)n2)nc1Cl] | 6-(5-amino-6-chloro-3- fluoropyridin-2-yl)-N²,N⁴-bis(3,3- difluorocyclobutyl-1,3,5-triazine- 2,4-diamine | 436.10 |

### Comparative Example 1

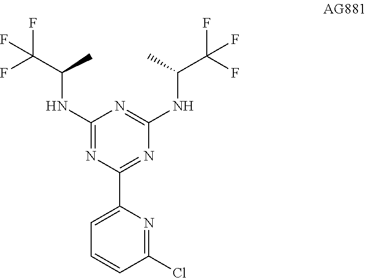 [SMILES: C[C@@H](Nc1nc(N[C@H](C)C(F)(F)F)nc(-c2cccc(Cl)n2)n1)C(F)(F)F]

The above-mentioned compounds were prepared according to compound 101 in WO 2015/003640A1.

### Kinetic Solubility Experiment

### I. Experimental Materials

### 1. Preparation of Solutions/Buffers at Different pH Values

Solution A: 17.42 g of K₂HPO₄ was weighed and dissolved in 1 L of ultrapure water.
Solution B: 13.65 g of KH₂PO₄ was weighed and dissolved in 1 L of ultrapure water.

| pH value | Preparation method: |
|---|---|
| 7.4 | An A + B solution in an appropriate ratio was |
| | adjusted to pH 7.4 |
| 5.0 | An appropriate amount of a buffer at pH 7.4 was |
| | taken and adjusted to pH 5.0 by adding a certain |
| | volume of an HCl solution |
| 3.0 | A certain volume of solution B was adjusted to |
| | pH 3.0 by adding an appropriate amount of an |
| | HCl solution |
| 1.0 | 9.00 mL of an HCl solution was diluted in 1 L |
| | of ultrapure water |

### 2. Main Experimental Reagents and Consumables

DMSO, methanol, acetonitrile, ultrapure water, EP tube, 96-well plate, etc.

### 3. Experimental Instruments

Thermostatic oscillator, centrifuge, LC-MS/MS.

### II. Experimental Steps

### 1. Preparation of Stock Solution

A certain mass of substance to be tested was accurately weighed and dissolved in a certain volume of DMSO to obtain a 20 mM or 10 mM stock solution.

### 2. Preparation of Internal Standard Stop Solution

An acetonitrile stopping solution containing 1 ng/mL of labetalol and glibenclamide was prepared.

3. Incubation System (n=3)

| | Injection | Buffer | Final |
|---|---|---|---|
| Stock solution | volume | volume | concentration |
| Compound to | 1.5 μL | 148.6 μL | 200 μM |
| be tested | | | |
| (20 mM) | | | |

The above-mentioned solutions were placed in a 96-well plate, respectively and shaken in a thermostatic oscillator for 2 hours (37° C., 150 rpm).

4. Control solution: 200 μM control solution of the substance to be tested was prepared in methanol.
5. Sample treatment

At a predetermined time, the 96-well plate was placed in a centrifuge and centrifuged at 4100 rpm for 15 minutes. A certain volume of the supernatant and a certain volume of control solution were respectively taken into 19 times volume of water containing 70% acetonitrile that has been previously added. The mixture was uniformly mixed, diluted 20 times with an internal standard stop solution and shaken in a thermostatic oscillator at 700 rpm at room temperature for 5 min. Further dilution may be necessary depending on the sensitivity of the compound, and sample analysis was performed by LC-MS/MS.

### III. Data Processing

The ratio of compound peak area to internal standard peak area was used for calculation. According to the concentration relationship between the compound in the buffer and the control solution, the calculated concentration was the kinetic solubility, and the calculation formula was as follows:

Solubility(μg/mL)=mass spectral peak area of the compound to be tested in buffers at different pHs×200μM×molecular weight of the compound to be tested(MW)/mass spectral peak area of the compound to be tested in methanol/1000;

The test results were as shown in table 1.

TABLE 1

| | Solubility (μg/mL) | | | |
|---|---|---|---|---|
| Compound | pH = 1.0 | pH = 3.0 | pH = 5.0 | pH = 7.4 |
| Comparative | 39.6 | 1.6 | 1.4 | 0.8 |
| example 1 | | | | |
| Example 5 | 86.6 | 53.3 | 59.5 | 48.1 |

It can be seen therefrom that, the compounds of the present invention have good kinetic solubility at different pH conditions, and the kinetic solubility of the compounds of the present invention at different pH conditions is much greater than that of the compound of comparative example 1, and is essentially free of pH-dependent phenomena.

### PHARMACOLOGICAL EXPERIMENTS

### Example A: Enzyme Activity Inhibition Assay

The inhibitory ability of the compounds on the enzyme activities of IDH1^{R132H} and IDH2^{R140Q}was detected and expressed as the half inhibitory concentration (IC₅₀) value. AG-881 was used as a positive control compound. In this experiment, compounds were screened on enzymes IDH1^{R132H} and IDH2^{R140Q} using a fluorescence-based method (initial concentration: 10000 nM, 3-fold dilution, 10 concentrations, and single-well detection).
(1) Preparation of 1×assay buffer.
(2) Preparation of compound concentration gradient: the compounds to be tested were assayed at an initial concentration of 10000 nM, with 3-fold dilution and 10 concentrations, and diluted into 200-fold the final concentration in 100% DMSO solution in a 384-well plate.
(3) 250 nL of DMSO and 250 nL of diluted compounds were transferred to a reaction plate with Echo 550.
(4) IDH2^{R140Q} enzyme detection: 25 μL of an IDH2^{R140Q} enzyme solution was added, and the mixture was incubated for 60 minutes; then 25 μL of a substrate solution was added, and the mixture was incubated for 150 minutes; then 25 μL of a detection buffer was added, and the mixture was shaken for 1 minute and incubated for 60 minutes; and finally, Synergy 2, Ex544/Em590 cutoff 590 was used for reading.
(5) IDH1^{R132H} enzyme detection: 40 μL of a mixture solution of an IDH1^{R132H} enzyme at the 1.25-fold final concentration and NADPH was added, and the mixture was incubated for 60 minutes; then 10 μL of a substrate solution at the 5-fold final concentration was added, and the mixture was incubated for 90 minutes; then 25 μL of a detection buffer at the 3-fold final concentration was added, and the mixture was shaken for 1 minute. and finally, Synergy 2, Ex544/Em590 cutoff 590 was used for reading.
(8) Inhibition rate calculation, formula: % Inhibition=(RFU_sample−RFU_min)/(RFU_max−RFU_min)*100%, wherein RFU-sample represents the fluorescence intensity of a sample; RFU-min represents the average value of negative control wells and represents the fluorescence intensity with an enzyme; and RFU-max represents the average value of positive control wells and represents the fluorescence intensity without an enzyme. The curve fitting was performed using Graphpad Prism software to obtain IC₅₀ values, and the test results were as shown in table 2.

### Example B: 2-HG Assay in Cells

The compounds were detected for the inhibition of 2-HG production in the U87 cell line stably transfected with IDH1-R132H and the culture supernatant of TF-1 cells stably transfected with IDH2-R140Q.
(1) The cultured cells were collected by centrifugation, then suspended in a cell culture solution, counted, and seeded in a 96-well cell culture plate at 160 μL of cell suspension/well (20000 cells/well for U87-IDH1-R132H; 10000 cells/well for TF-1-IDH2-R140Q).
(2) A 96-well plate dilution was taken, and the compounds to be tested were diluted in a gradient manner in DMSO and a cell culture solution; and then 40 μL of the gradient-diluted compounds to be tested were added to the 96-well plate on which cells have been plated. U87-IDH1-R132H cells had an initial concentration of 2 μM and subjected to 3-fold gradient dilution; TF-1-IDH2-R140Q cells had an initial concentration of 10 μM and subjected to 3-fold gradient dilution (the final DMSO concentration was 0.2%); and the final volume was 200 μL/well. Finally, the plate was cultured in a 37° C. incubator containing 5% CO₂.
(3) After the compounds were incubated with the cells for 72 hours, the cell culture plate was taken out and centrifuged at 1500 rpm for 5 minutes, and then 100 μL of the medium supernatant was aspirated for 2-HG detection.
(4) 2-HG detection: the cell supernatant was diluted with purified water, 30 μL of the diluted sample was taken into a 1.5 ml centrifuge tube, and 200 μL of the internal standard solution was added. The mixture was mixed uniformly by vortex and centrifuged at 3200 rpm at 4° C. for 15 minutes. 100 μL of the supernatant solution was diluted with water at a 1:1 ratio and then detected by LC-MS. Analyte Peak Area was used to represent the 2-HG levels.
(5) Inhibition rate calculation, formula: % Inhibition=(1−Analyte Peak Area_sample/Analyte Peak Area_max)*100%, wherein Analyte Peak Area_sample represents the Analyte Peak Area of the sample to which the compound was added; Analyte Peak Area_max represents the average value of Analyte Peak Area of positive control wells (representing the absence of the compound). The curve fitting was performed using Graphpad Prism software to obtain IC₅₀ values, and the test results were as shown in table 2.

The results of the enzyme activity inhibition assay and the 2-HG inhibition assay in cells are expressed as IC₅₀, wherein “A” represents “IC₅₀≤50 nM”; “B” represents “50 nM<IC₅₀<100 nM”; “C” represents “100 nM<IC₅₀≤1000 nM”; “D” represents “IC₅₀>1000 nM”. Note: “-” represents “not tested”.

TABLE 2

| | Enzyme activity | | 2-HG in medium |
|---|---|---|---|
| | (IC₅₀, nM) | | (IC₅₀, nM) |
| Example | IDH1^{R132H} | IDH2^{R140Q} | (IDH1^{R132H}) |
| Example 1 | 143 | 128 | 26 |
| Example 2 | 22 | — | 17 |
| Example 3 | D | — | D |
| Example 4 | D | — | D |
| Example 5 | 8 | 190 | 0.5 |
| Example 6 | C | — | B |
| Example 7 | D | C | B |
| Example 8 | D | C | C |
| Example 9 | D | — | D |
| Example 10 | D | — | C |
| Example 11 | D | — | C |
| Example 12 | A | — | A |
| Example 13 | D | — | C |
| Example 17 | A | — | A |
| Example 18 | B | B | D |
| Example 19 | B | B | D |
| Example 20 | D | — | — |
| Example 21 | C | — | — |
| Example 22 | D | — | — |
| Example 23 | C | C | A |
| Example 25 | C | — | C |

It can be seen therefrom that the compounds of the present invention have excellent enzymatic and cellular activities.

### Example C: Experimental Methods of Pharmacokinetic Detection in Dogs

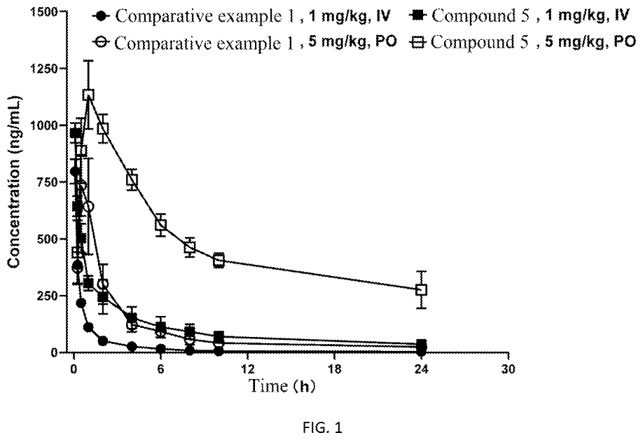

Adult beagle dogs (6-12 months of age) received a single dose of the compounds to be tested via intravenous administration (IV) or oral gavage administration (PO), respectively. In the IV single dose, 5% DMSO+5% Solutol+90% physiological saline was used as the excipient; the intravenous administration was carried out at a dose of 1 mg/kg (2 females and 2 males); and the venous blood collection time was 5 min, 15 min, 0.5 h, 1 h, 2 h, 4 h, 6 h, 8 h, 10 h and 24 h. In the PO single dose, 5% DMSO+5% Solutol+90% purified water was used as the excipient; the oral gavage administration was carried out at a dose of 5 mg/kg (2 females and 2 males); and the blood collection time was 15 min, 0.5 h, 1 h, 2 h, 4 h, 6 h, 8 h, 10 h and 24 h. Blood samples were collected in tubes containing EDTA anticoagulants and centrifuged at 4000 rpm at 4° C. for 10 min, and the supernatants were transferred to centrifuge tubes and stored at −20° C. For the detection, 30 μL of the plasma supernatant sample was taken, and 200 μL of an internal standard solution was added. The mixture was centrifuged at 3000 rpm for 10 minutes. Then, 100 μL of the supernatant solution was diluted with water at a 1:1 ratio and then injected with an injection volume of 5 μL. The concentration of the compounds to be tested in the plasma sample was analyzed by liquid chromatography-tandem mass spectrometry (LC-MS/MS). Plasma concentration-time data for individual animals were analyzed using Sciex Analyst software. The non-compartment model was introduced into the concentration analysis, and pharmacokinetic parameters (Cl_obs, Cₘₐₓ and AUCₗₐₛₜ) of the compounds to be tested were calculated using WinNonlin (version 4.1; pHarsight) software. The test results were shown in table 3, and the PK curves were shown in FIG. 1.

TABLE 3

| Mode of | | | | | |
|---|---|---|---|---|---|
| adminis- | | Dose | Cl_obs | Cₘₐₓ | AUCₗₐₛₜ |
| tration | Compound | (mg/kg) | (mL/min/kg) | (ng/mL) | (h · ng/mL) |
| IV | Comparative | 1 | 24.2 | / | 653 |
| | example 1 | | | | |
| | Compound 5 | 1 | 5.1 | / | 2627 |
| PO | Comparative | 5 | / | 658 | 2155 |
| | example 1 | | | | |
| | Compound 5 | 5 | / | 1160 | 11713 |

Note:
“/” indicates that the corresponding parameter is not detected.

It can be seen therefrom that the compounds of the present invention have good in vivo PK, such as higher Cₘₐₓ and oral drug absorption exposure AUCₗₐₛₜ; and the in vivo PK performance of the compounds of the present invention is superior to that of comparative example 1. Although the present invention has been fully described by way of embodiments thereof, it should be noted that various changes and modifications are obvious to those skilled in the art. Such changes and modifications should all fall within the scope of the appended claims of the present invention.

1. A compound represented by formula (VIb), or a pharmaceutically acceptable salt, solvate, chelate, non-covalent complex or prodrug thereof, wherein the compound represented by formula (VIb) has a structure of 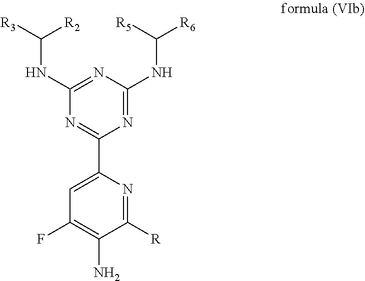
wherein
R is selected from halogen and C₁-C₄ haloalkyl;
R₂ and R₃ are independently selected from C₁-C₄ alkyl or C₃-C₆ cycloalkyl, wherein the C₁-C₄ alkyl and C₃-C₆ cycloalkyl are optionally substituted with one or more of hydrogen, halogen, oxo, —NO₂, —OH, —NH₂, —CN, C₁-C₄ alkyl, —O—C₁-C₄ alkyl, —NH(C₁-C₄ alkyl) or —N(C₁-C₄ alkyl)₂; or R₂ and R₃ together with the carbon atom to which they are attached form cyclopropane and cyclobutane; and the cyclopropane and cyclobutane are optionally substituted with one or more of hydrogen or halogen;
R₅ and R₆ are independently selected from C₁-C₄ alkyl or C₃-C₆ cycloalkyl, wherein the C₁-C₄ alkyl and C₃-C₆ cycloalkyl are optionally substituted with one or more of hydrogen, halogen, oxo, —NO₂, —OH, —NH₂, —CN, C₁-C₄ alkyl, —O—C₁-C₄ alkyl, —NH(C₁-C₄ alkyl) or —N(C₁-C₄ alkyl)₂; or R₅ and R₆ together with the carbon atom to which they are attached form cyclopropane and cyclobutane; and the cyclopropane and cyclobutane are optionally substituted with one or more of hydrogen or halogen.

2.-8. (canceled)

9. The compound according to claim 1, wherein R is selected from F, Cl, Br and CF₃.

10. The compound according to claim 1, wherein R is selected from Cl.

11. The compound according to claim 1, wherein R is selected from CF₃.

12. (canceled)

13. The compound according to claim 1, wherein R₂, R₃, R₅ and R₆ are independently selected from CF₃, CH₃ or cyclopropyl; further, R₂ is different from R₃, and R₅ is different from R₆.

14. The compound according to claim 1, wherein R₂ and R₅ are each independently selected from CH₃.

15. The compound according to claim 1, wherein R₃ and R₆ are each independently selected from CF₃ or cyclopropyl.

16.-28. (canceled)

29. The compound, or the pharmaceutically acceptable salt thereof according to claim 1, wherein the compound is selected from:
6-(5-amino-6-chloro-4-fluoropyridin-2-yl)-N²,N⁴-bis((R)-1,1,1-trifluoropropyl-2-yl)-1,3,5-triazine-2,4-diamine;
6-(5-amino-6-chloro-4-fluoropyridin-2-yl)-N²N⁴-bis((R)-1-cyclopropylethyl)-1,3,5-triazine-2,4-diamine;
6-(5-amino-6-chloro-4-fluoropyridin-2-yl)-N²,N⁴-bis(3,3-difluorocyclobutyl)-1,3,5-triazine-2,4-diamine;
6-(5-amino-4-fluoro-6-(trifluoromethyl)pyridin-2-yl)-N²,N⁴-bis((R) 1,1,1-trifluoroprop-2-yl)-1,3,5-triazine-2,4-diamine;
6-(5-amino-4-fluoro-6-(trifluoromethyl)pyridin-2-yl)-N²,N⁴-bis((R)-1-cyclopropylethyl)-1,3,5-triazine-2,4-diamine;
6-(5-amino-4-fluoro-6-(trifluoromethyl)pyridin-2-yl)-N²,N⁴-bis((3,3-difluorocyclobutyl)-1,3,5-triazine-2,4-diamine;
6-(5-amino-6-chloro-4-fluoropyridin-2-yl)-N²,N⁴-bis((R)-1,1-difluoroprop-2-yl)-1,3,5-triazine-2,4-diamine;
6-(5-amino-4,6-difluoropyridin-2-yl)-N²,N⁴-bis((R)-1,1,1-trifluoroprop-2-yl)-1,3,5-triazine-2,4-diamine;
6-(6-chloro-4-fluoro-5-aminopyridin-2-yl)-N²,N⁴-dicyclopropyl-1,3,5-triazine-2,4-diamine;
or
6-(5-amino-6-chloro-4-fluoropyridin-2-yl)-N²,N⁴-diisopropyl-1,3,5-triazine-2,4-diamine.

30. A pharmaceutical composition, wherein the pharmaceutical composition comprises a therapeutically effective amount of at least one compound according to claim 1 and at least one pharmaceutically acceptable excipient.

31.-36. (canceled)

37. A method for treating and/or preventing an IDH1 and IDH2-mediated disease by administering a therapeutically effective amount of at least one compound according to claim 1 or a pharmaceutical composition comprising a therapeutically effective amount of at least one compound according to claim 1 and at least one pharmaceutically acceptable excipient to a subject to be treated.

38. The method according to claim 37, wherein the IDH1 and IDH2-mediated disease is cancer.

39. A method for treating cancer, wherein the method comprises administering a therapeutically effective amount of at least one compound according to claim 1 or a pharmaceutical composition comprising a therapeutically effective amount of at least one compound according to claim 1 and at least one pharmaceutically acceptable excipient to a subject to be treated.

40. The method according to claim 39, wherein the method involves a method for treating cancer characterized by the presence of mutant IDH1 and IDH2, comprising administering a therapeutically effective amount of the compound, pharmaceutically acceptable salt, crystal, solvate or prodrug thereof, or the pharmaceutical composition to a subject to be treated, wherein the cancer is selected from brain glioma, melanoma, papillary thyroid tumor, cholangiocarcinoma, lung cancer, breast cancer, sarcoma, glioma, glioblastoma multiforme, acute myeloid leukemia and non-Hodgkin's lymphoma.

41. The method according to claim 37, wherein the subject to be treated is human.

42. The method according to claim 40, wherein the subject to be treated is human.

43. The method according to claim 38, wherein the cancer is selected from brain glioma, melanoma, papillary thyroid tumor, cholangiocarcinoma, lung cancer, breast cancer, sarcoma, glioma, glioblastoma multiforme, acute myeloid leukemia and non-Hodgkin's lymphoma.
